(19) United States
(12) Patent Application Publication
(10) Pub. No.: US 2026/0284402 A1
(43) Pub. Date: Sep. 24, 2026

(54) METHODS AND SYSTEMS FOR STIMULATION CONTROL BASED ON dCSF THICKNESS MONITORING

(71) Applicant: Boston Scientific Neuromodulation Corporation, Valencia, CA (US)

(72) Inventors: Luis Arturo Herrera Vega, Valencia, CA (US); Akhil Srinivasan, Porter Ranch, CA (US); Andrew James Haddock, Los Angeles, CA (US); Daniel Antonio Vargas Campos, Alajuela (CR); Sarvani Grandhe, Valencia, CA (US); Tianhe Zhang, Studio City, CA (US)

(21) Appl. No.: 19/085,630

(22) Filed: Mar. 20, 2025

Related U.S. Application Data
(60) Provisional application No. 63/571,131, filed on Mar. 28, 2024.

(51) Int. Cl. A61N 1/36; A61N 1/372

(52) U.S. Cl. CPC A61N 1/36139; A61N 1/3614; A61N 1/37247

(57) ABSTRACT

This document discusses a neurostimulation device. The neurostimulation device includes a stimulation circuit configured to deliver electrical neurostimulation energy to a subject when coupled to an implantable lead, a physiological sensor circuit to produce a sensed physiological signal, a memory to store dorsal cerebrospinal fluid (dCSF) thickness data for the subject, and a control circuit operatively coupled to the stimulation circuit, the physiological sensor circuit, and the memory. The control circuit is configured to determine physiological information of the subject using the sensed physiological signal, determine a value of dCSF thickness according to the physiological information and the stored dCSF thickness data, and set a parameter of the electrical neurostimulation therapy according to the determined value of dCSF thickness.

### CLAIM OF PRIORITY

This application claims the benefit of U.S. Provisional Application No. 63/571,131, filed on Mar. 28, 2024, which is hereby incorporated by reference in its entirety.

### TECHNICAL FIELD

This document relates generally to medical devices and more particularly to a system for neurostimulation.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., SCS) the efficacy of the neurostimulation for the patient may depend on an intricate balance of stimulation location coupled with the programmed stimulation waveform.

### SUMMARY

Electrical neurostimulation energy can be delivered in the form of electrical neurostimulation pulses to treat a neurological condition of the patient. The pulses can be delivered to a neurostimulation target using an implantable stimulation lead. The lead can have multiple electrodes and may be configurable into many electrode configurations. The neurostimulation energy can be customized for a specific patient in a clinical setting. However, the distance and direction of the neurostimulation target relative to the electrodes may change with movement of the patient.

A first Example (Example 1) includes subject matter (such as a neurostimulation device) comprising a stimulation circuit configured to deliver electrical neurostimulation energy to a subject when coupled to an implantable lead, a physiological sensor circuit to produce a sensed physiological signal, a memory to store dorsal cerebrospinal fluid (dCSF) thickness data for the subject, and a control circuit operatively coupled to the stimulation circuit, the physiological sensor circuit, and the memory. The control circuit is configured to determine physiological information of the subject using the sensed physiological signal, determine a value of dCSF thickness according to the physiological information and the stored dCSF thickness data, and set a parameter of the electrical neurostimulation therapy according to the determined value of dCSF thickness.

In Example 2, the subject matter of Example 1 optionally includes a physiological sensor circuit that is a posture sensing circuit and the sensed physiological signal is a sensed posture signal produced by the posture sensing circuit. The subject matter also optionally includes a control circuit configured to determine position information of the subject using the sensed posture signal, and determine the value of dCSF thickness using the determined position and the stored dCSF thickness data.

In Example 3, the subject matter of one or both of Examples 1 and 2 optionally includes an impedance sensing circuit configured to produce a sensed electrical impedance signal, and a control circuit configured to produce electrical impedance image data for the subject at multiple anatomical locations using sensed electrical impedance signals, determine dCSF thickness data for the anatomical locations, and store the dCSF thickness data for the subject in the neurostimulation device in association with position information of the subject.

In Example 4, the subject matter of Example 3 optionally includes a user interface operatively coupled to the control circuit, and a control circuit configured to receive the position information and one or more electrical impedance imaging parameters via the user interface.

In Example 5, the subject matter of Example 4 optionally includes a control circuit configured to determine a value of variation of dCSF thickness of the subject with posture using the dCSF thickness data, and present a recommendation of using dCSF thickness to adjust the neurostimulation therapy based on the variation of the dCSF thickness of the subject.

In Example 6, the subject matter of one or any combination of Examples 1-5 optionally includes a physiological sensor circuit that is a signal sensing circuit configured to produce sensed Evoked Compound Action Potential (ECAP) signals when coupled to the implantable lead. The subject matter also optionally includes a control circuit configured to initiate sensing of an ECAP signal at an anatomical location of the subject and determine ECAP signal data using the sensed ECAP signal, and determine the value of dCSF thickness using the determined ECAP signal data and the stored dCSF thickness data.

In Example 7, the subject matter of Example 6 optionally includes impedance sensing circuit configured to produce a sensed electrical impedance signal and a control circuit configured to produce electrical impedance image data for the subject at multiple anatomical locations using sensed electrical impedance signals, determine dCSF thickness data for the anatomical locations, and store the dCSF thickness data for the subject in the neurostimulation device in association with the ECAP signal data.

In Example 8, the subject matter of one or any combination of Examples 1-7 optionally includes an impedance sensing circuit configured to produce a sensed electrical impedance signal, and a control circuit including processing circuitry configured to produce electrical impedance image data for the subject at multiple anatomical locations of the subject using electrical impedance tomography (EIT), and determine the dCSF thickness data for the anatomical locations using the electrical impedance image data.

Example 9 includes subject matter (such as a computer-implemented method of operating a neurostimulation device) or can optionally be combined with one or any combination of Examples 1-8 to include such subject matter, comprising sensing a physiological signal using a physiological sensor circuit, wherein the physiological signal includes physiological information of a subject, determining dorsal cerebrospinal fluid (dCSF) thickness for the subject according to the physiological information using dCSF thickness data stored in the neurostimulation device, and setting a parameter of the neurostimulation therapy according to determined dCSF thickness.

In Example 10, the subject matter of Example 9 optionally includes sensing a position of the subject using a posture sensing circuit of the neurostimulation device, and determining the dCSF thickness according to the position and the stored dCSF thickness data.

In Example 11, the subject matter of Example 10 optionally includes producing electrical impedance image data for the subject at multiple anatomical locations and multiple positions of the subject using electrical impedance tomography (EIT) or electrode impedance spectroscopy (EIS), determining dCSF thickness data for the anatomical locations and positions of the subject using the electrical impedance image data, and storing the dCSF thickness data for the subject in the neurostimulation device in association with patient position.

In Example 12, the subject matter of one or any combination of Examples 9-11 optionally includes sensing posture and movement of the subject using an accelerometer circuit of the neurostimulation device, and determining the dCSF thickness according to the sensed posture and movement and the stored dCSF thickness data.

In Example 13, the subject matter of one or any combination of Examples 9-12 optionally includes sensing an Evoked Compound Activity Potential (ECAP) signal of the subject using a signal sensing circuit of the neurostimulation device, and determining the dCSF thickness according to stored ECAP signal data and the stored dCSF thickness data.

In Example 14, the subject matter of Example 13 optionally includes sensing ECAP signals at multiple anatomical locations of the subject, determining ECAP signal data for the sensed ECAP signals and storing the ECAP signal data in the neurostimulation device, determining electrical impedance image data for the subject at the multiple anatomical locations, and determining dCSF thickness data for the multiple anatomical locations using the electrical impedance image data and storing the dCSF thickness data for the subject in the neurostimulation device in association with the ECAP signal data.

In Example 15, the subject matter of one or any combination of Examples 9-14 optionally includes producing electrical impedance image data for the subject at multiple anatomical locations of the subject, determining the dCSF thickness data for the anatomical locations using the electrical impedance image data, determining a variation of dCSF thickness of the subject using the dCSF thickness data, and producing a recommendation of adjusting the neurostimulation therapy based on the variation of the dCSF thickness of the subject.

In Example 16, the subject matter of one or any combination of Examples 9-15 optionally includes setting one or both of power of the neurostimulation therapy and direction of the neurostimulation therapy according to determined dCSF thickness.

Example 17 includes subject matter (such as a neurostimulation programming device) or can optionally be combined with one or any combination of Examples 1-16 to include such subject matter, comprising a communication circuit configured to communicate information with a neurostimulation device, an electrical impedance sensing circuit configured to produce an electrical impedance signal when operatively coupled to electrodes, and processing circuitry. The processing circuitry is configured to produce electrical impedance image data for a patient at multiple anatomical locations of the patient using one of electrical impedance tomography (EIT) or electrode impedance spectroscopy (EIS), determine dorsal cerebrospinal fluid (dCSF) thickness data for the multiple anatomical locations using the electrical impedance image data, and communicate the dCSF thickness data to a neurostimulation device.

In Example 18, the subject matter of Example 17 optionally includes a user interface, and processing circuitry configured to receive position information of the patient via the user interface, determine the dCSF thickness data for multiple patient positions, and store the dCSF thickness data for the patient in memory of the neurostimulation device in association with patient position.

In Example 19, the subject matter of one or both of Examples 17 and 18 optionally includes a stimulation circuit configured to deliver electrical neurostimulation energy to a subject when coupled to the electrodes, a signal sensing circuit configured to sense evoked potential signals when coupled to the electrodes, and processing circuitry configured to initiate delivery of neurostimulation to the subject that produces an Evoked Compound Activity Potential (ECAP), record sensed ECAP signals at the multiple anatomical locations resulting from the neurostimulation, determine the dCSF thickness data for the multiple anatomical locations, and store ECAP signal data in memory of the neurostimulation device in association with the dCSF thickness data.

In Example 20, the subject matter of one or any combination of Examples 17-19 optionally includes processing circuitry configured to store, in memory of the neurostimulation device, parameters of electrical neurostimulation therapy provided by the neurostimulation device in association with determined values of dCSF thickness.

These non-limiting Examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of a neurostimulation system.

FIG. 2 is an illustration of portions of another example of a neurostimulation system.

FIG. 3 is an illustration of an example of an implantable pulse generator (IPG) and an implantable lead system.

FIG. 4 is an illustration of another example of an implantable stimulation lead.

FIGS. 5A-5H are illustrations of an example of electrodes of a stimulation lead.

FIG. 6 is an illustration of another example of an implantable lead system.

FIG. 7 is an illustration of an example of an implantable lead system and spinal cord anatomy.

FIG. 8 is a block diagram of portions of an example of a neurostimulation device for providing electrical neurostimulation to a patient.

FIG. 9 is a flow diagram of an example of a method to operate a neurostimulation device.

FIG. 10 is a block diagram of portions of another example of a medical device.

FIG. 11 is a simplified diagram of producing electrical impedance image data.

FIG. 12 is an example of an image produced using electrical impedance image data.

FIG. 13 is an illustration of an example of a user interface display of a medical device.

FIGS. 14-17 are illustrations of additional examples of a user interface display of a medical device.

FIG. 18 is an illustration of a stimulation cuff that includes multiple electrodes for stimulation and sensing.

FIGS. 19-21 are examples of a user interface display to assist the user in producing impedance imaging data of a peripheral nerve.

FIGS. 22-23 are examples of a user interface display to assist the user in selecting electrodes for neurostimulation based on impedance imaging data.

FIGS. 24-26 are examples of a user interface display to receive user selections of neurostimulation of a peripheral nerve target.

FIG. 27 is an illustration of an example of a grid for electrode layout for impedance imaging.

FIGS. 28-32 are illustrations of examples of stimulation patterns to produce impedance image data for imaging of a peripheral nerve.

FIGS. 33-34 are timing diagrams of subthreshold stimulation to produce impedance image data.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS, SCS, PNS, FES, Occipital Nerve Stimulation (ONS), Sacral Nerve Stimulation (SNS), and Vagus Nerve Stimulation (VNS) therapies.

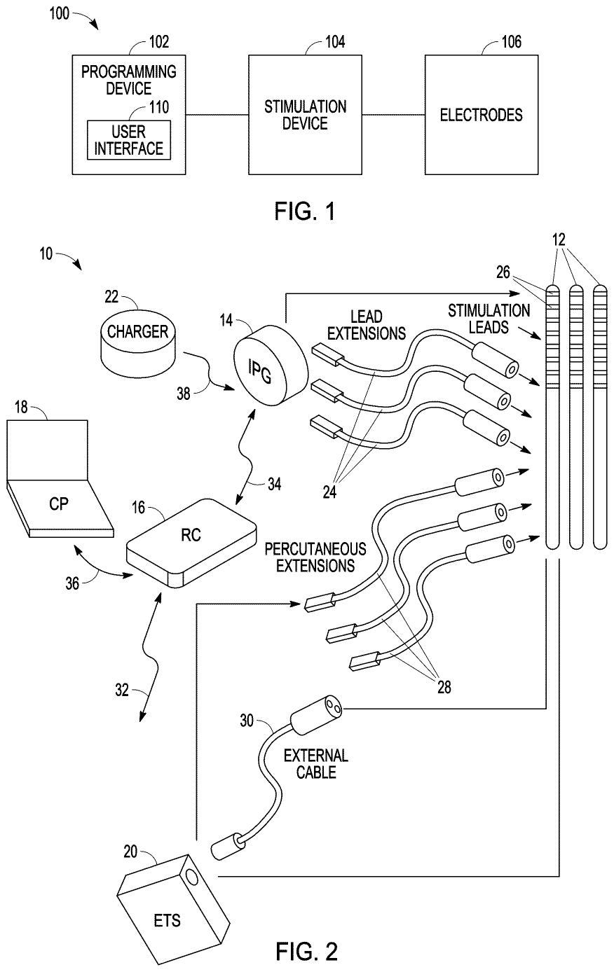

FIG. 1 illustrates an example of portions of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using multiple stimulation parameters, such as stimulation parameters specifying a pattern of the electrical pulses and a selection of electrodes through which each of the electrical pulses is delivered. In various embodiments, at least some of the stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In this document, a “user” includes a physician or other clinician or caregiver who treats the patient using system 100; a “patient” includes a person who receives or is intended to receive neurostimulation delivered using system 100. In various embodiments, the patient can be allowed to adjust his or her treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for SCS applications. The SCS configurations include various features that may simplify the task of the user in programming the stimulation device 104 for delivering SCS to the patient, such as the features discussed in this document.

FIG. 2 is an illustration of portions of another example of a neurostimulation system 10 that includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

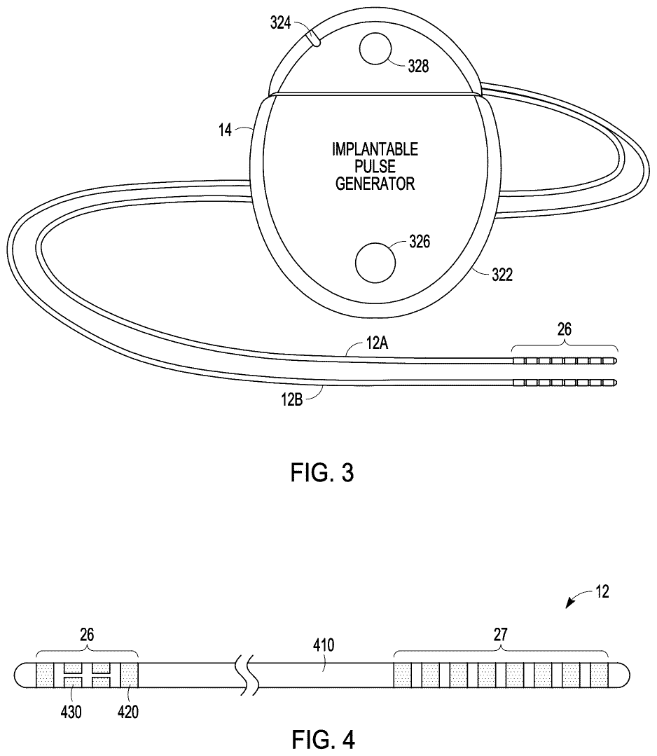

FIG. 3 is an illustration of an example of an IPG 14 (e.g., IPG 14 in FIG. 2) and an implantable lead system that includes stimulation leads (e.g., stimulation leads 12 in FIG. 2). The IPG 14 can be used as stimulation device 104 in FIG. 1. As illustrated in FIG. 3, IPG 14 that can be coupled to implantable leads 12A and 12B at a proximal end of each lead. The distal end of each lead includes electrical contacts or electrodes 26 for contacting a tissue site targeted for electrical neurostimulation. As illustrated in FIG. 3, leads 12A and 12B each include 8 electrodes 26 at the distal end. The number and arrangement of leads 12A and 12B and electrodes 26 as shown in FIGS. 2 and 3 are only examples, and other numbers and arrangements are possible. In various examples, the lead electrodes 26 are ring electrodes. In various examples the lead electrodes 26 include one or more segmented electrodes.

The IPG 14 can include a hermetically sealed IPG case 322 to house the electronic circuitry of IPG 14. IPG 14 can include an electrode 326 formed on IPG case 322. IPG 14 can include an IPG header 324 for coupling the proximal ends of leads 12A and 12B. IPG header 324 may optionally also include an electrode 328. One or both of electrodes 326 and 328 may be used as a reference electrode.

The implantable leads and electrodes may be configured by shape and size to provide electrical neurostimulation energy to a neuronal target at or near the spinal cord of the subject. Neurostimulation energy can be delivered in a monopolar (also referred to as unipolar) mode using electrode 326 or electrode 328 and one or more electrodes selected from electrodes 26. Neurostimulation energy can be delivered in a bipolar mode using a pair of electrodes of the same lead (lead 12A or lead 12B). Neurostimulation energy can be delivered in an extended bipolar mode using one or more electrodes of a lead (e.g., one or more electrodes of lead 12A) and one or more electrodes of a different lead (e.g., one or more electrodes of lead 12B).

After implantation, a clinician will program the neurostimulation device 400 using a CP 18, remote control, or other programming device. The electronic circuitry of IPG 14 can include a stimulation control circuit that controls delivery of the neurostimulation energy. The stimulation control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

FIG. 4 is a schematic side view of an embodiment of an electrical stimulation lead usable for SCS or other neurostimulation. FIG. 4 illustrates a stimulation lead 12 with electrodes 26 disposed at least partially about a circumference of the lead 12 along a distal end portion of the lead and terminals 27 disposed along a proximal end portion of the lead. The lead 12 can be implanted near or within the desired portion of the body to be stimulated (e.g., the spinal cord, brain, or other body organs or tissues).

The stimulation lead 12 can include stimulation electrodes, recording electrodes, or both. In at least some embodiments, the lead 12 is rotatable so that the stimulation electrodes can be aligned with the target neurons after the neurons have been located using the recording electrodes. Stimulation electrodes may be disposed on the circumference of the lead 12 to stimulate the target neurons. Stimulation electrodes may be ring-shaped so that current projects from each electrode equally in every direction from the position of the electrode along a length of the lead 12. In the embodiment of FIG. 4, two of the electrodes 420 are ring electrodes 420. Ring electrodes typically do not enable stimulus current to be directed from only a limited angular range around of the lead. Segmented electrodes 430, however, can be used to direct stimulus current to a selected angular range around the lead. When segmented electrodes 430 are used in conjunction with an IPG 14 that delivers constant current stimulus, current steering can be achieved to more precisely deliver the stimulus to a position around an axis of the lead (e.g., radial positioning around the axis of the lead). To achieve current steering, segmented electrodes can be utilized in addition to, or as an alternative to, ring electrodes.

The lead 12 includes a lead body 410, terminals 27, and one or more ring electrodes 420 and one or more sets of segmented electrodes 430 (or any other combination of electrodes). The lead body 410 can be formed of a biocompatible, non-conducting material such as, for example, a polymeric material. Suitable polymeric materials include, but are not limited to, silicone, polyurethane, polyurea, polyurethaneurea, polyethylene, or the like. Once implanted in the body, the lead 12 may be in contact with body tissue for extended periods of time. In at least some embodiments, the lead 12 has a cross-sectional diameter of no more than 1.5 millimeters (1.5 mm) and may be in the range of 0.5 to 1.5 mm. In at least some embodiments, the lead 12 has a length of at least 10 centimeters (10 cm) and the length of the lead 12 may be in the range of 10 to 70 cm.

The electrodes 26 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used or unused (OFF). When the electrode is used, the electrode can be used as an anode or cathode and carry anodic or cathodic current. In some instances, an electrode might be an anode for a period of time and a cathode for a period of time.

Spinal cord stimulation leads 12 and other leads may include one or more sets of segmented electrodes. Segmented electrodes may provide for superior current steering than ring electrodes because target structures may not be symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue.

Any number of segmented electrodes 430 may be disposed on the lead body 410 including, for example, anywhere from one to sixteen or more segmented electrodes 430. It will be understood that any number of segmented electrodes 430 may be disposed along the length of the lead body 410. A segmented electrode 430 typically extends only 75%, 67%, 60%, 50%, 40%, 33%, 25%, 20%, 17%, 15%, or less around the circumference of the lead.

The segmented electrodes 430 may be grouped into sets of segmented electrodes, where each set is disposed around a circumference of the lead 12 at a particular longitudinal portion of the lead 12. The lead 12 may have any number segmented electrodes 430 in a given set of segmented electrodes. The lead 12 may have one, two, three, four, five, six, seven, eight, or more segmented electrodes 430 in a given set. In at least some embodiments, each set of segmented electrodes 430 of the lead 12 contains the same number of segmented electrodes 430. The segmented electrodes 430 disposed on the lead 12 may include a different number of electrodes than at least one other set of segmented electrodes 430 disposed on the lead 12. The segmented electrodes 430 may vary in size and shape. In some embodiments, the segmented electrodes 430 are all of the same size, shape, diameter, width or area or any combination thereof. In some embodiments, the segmented electrodes 430 of each circumferential set (or even all segmented electrodes disposed on the lead 12) may be identical in size and shape.

Each set of segmented electrodes 430 may be disposed around the circumference of the lead body 410 to form a substantially cylindrical shape around the lead body 410. The spacing between individual electrodes of a given set of the segmented electrodes may be the same, or different from, the spacing between individual electrodes of another set of segmented electrodes on the lead 12. In at least some embodiments, equal spaces, gaps or cutouts are disposed between each segmented electrode 430 around the circumference of the lead body 410. In other embodiments, the spaces, gaps or cutouts between the segmented electrodes 430 may differ in size, or cutouts between segmented electrodes 430 may be uniform for a particular set of the segmented electrodes 430 or for all sets of the segmented electrodes 430. The sets of segmented electrodes 430 may be positioned in irregular or regular intervals along a length the lead body 410.

Conductor wires (not shown) that attach to the ring electrodes 420 or segmented electrodes 430 extend along the lead body 410. These conductor wires may extend through the material of the lead 12 or along one or more lumens defined by the lead 12, or both. The conductor wires couple the electrodes 420, 430 to the terminals 27.

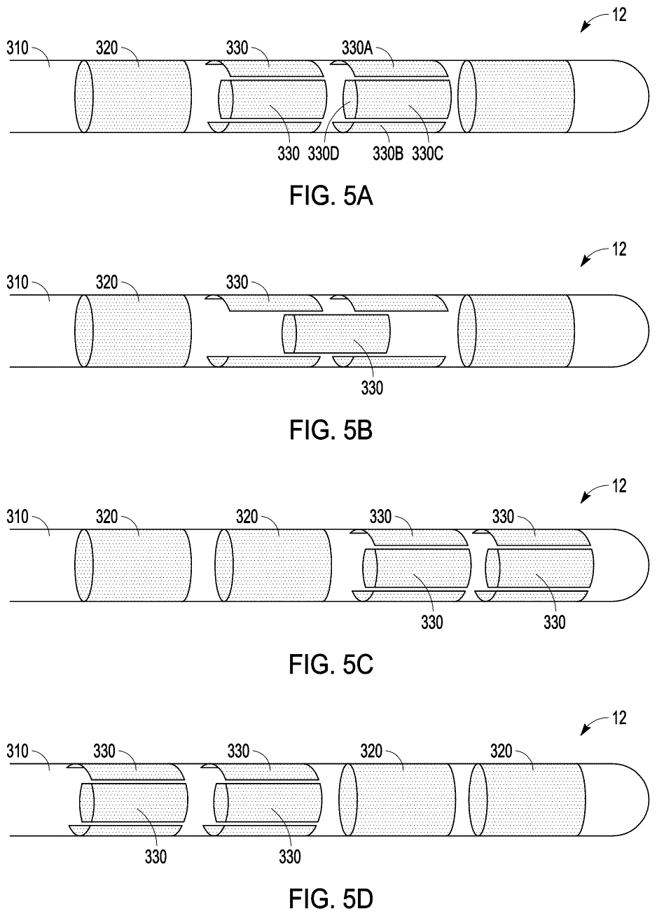

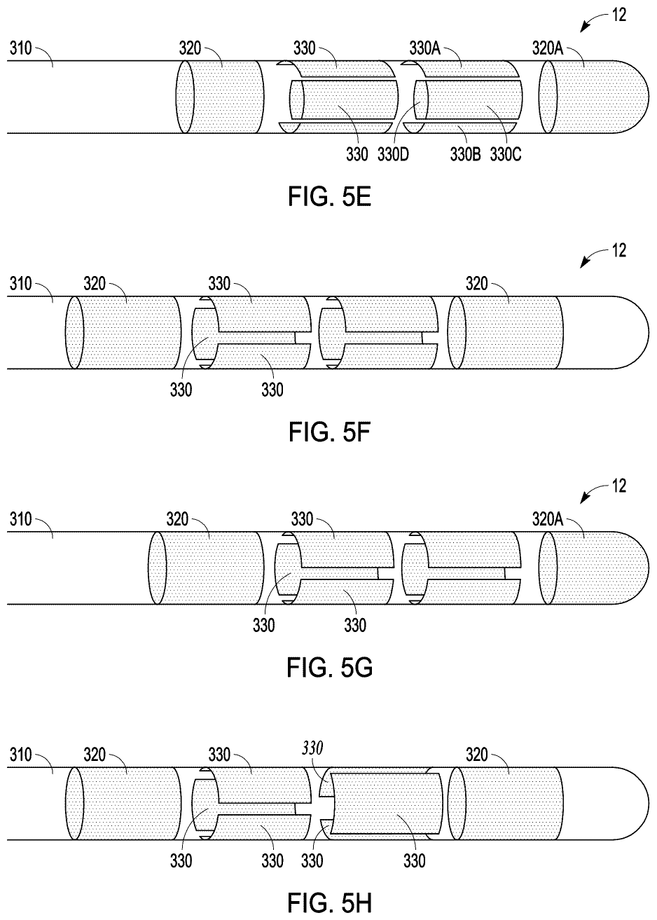

FIGS. 5A-5H are illustrations of different embodiments of leads 12 with segmented electrodes 330, optional ring electrodes 320 or tip electrodes 320a, and a lead body 310. The sets of segmented electrodes 330 each include either two (FIG. 5B), three (FIGS. 5E-5H), or four (FIGS. 5A, 5C, and 5D) or any other number of segmented electrodes including, for example, three, five, six, or more. The sets of segmented electrodes 330 can be aligned with each other (FIGS. 5A-5G) or staggered (FIG. 5H).

When the lead 12 includes both ring electrodes 320 and segmented electrodes 330, the ring electrodes 320 and the segmented electrodes 330 may be arranged in any suitable configuration. For example, when the lead 12 includes two ring electrodes 320 and two sets of segmented electrodes 330, the ring electrodes 120 can flank the two sets of segmented electrodes 330 (see e.g., FIGS. 4, 5A, and 5E-5H, ring electrodes 320 and segmented electrode 330). Alternately, the two sets of ring electrodes 320 can be disposed proximal to the two sets of segmented electrodes 330 (see e.g., FIG. 5C, ring electrodes 320 and segmented electrode 330), or the two sets of ring electrodes 320 can be disposed distal to the two sets of segmented electrodes 330 (see e.g., FIG. 5D, ring electrodes 320 and segmented electrode 330). One of the ring electrodes can be a tip electrode (see e.g., tip electrode 320a of FIGS. 5E and 5G). It will be understood that other configurations are possible as well (e.g., alternating ring and segmented electrodes, or the like).

By varying the location of the segmented electrodes 330, different coverage of the target tissue may be selected. For example, the electrode arrangement of FIG. 5C may be useful if the physician anticipates that the neural target will be closer to a distal tip of the lead body 310, while the electrode arrangement of FIG. 5D may be useful if the physician anticipates that the neural target will be closer to a proximal end of the lead body 310.

Any combination of ring electrodes 320 and segmented electrodes 330 may be disposed on the lead 12. For example, the lead 12 may include a first ring electrode 320, two sets of segmented electrodes; each set formed of four segmented electrodes 330, and a final ring electrode 320 at the end of the lead. This configuration may simply be referred to as a 1-4-4-1 configuration (FIGS. 5A and 5E, ring electrodes 320 and segmented electrode 330). It may be useful to refer to the electrodes with this shorthand notation. Thus, the embodiment of FIG. 5C may be referred to as a 1-1-4-4 configuration, while the embodiment of FIG. 5D may be referred to as a 4-4-1-1 configuration. The embodiments of FIGS. 5F, 5G, and 5H can be referred to as a 1-3-3-1 configuration. Other electrode configurations include, for example, a 2-2-2-2 configuration, where four sets of segmented electrodes are disposed on the lead, and a 4-4 configuration, where two sets of segmented electrodes, each having four segmented electrodes 330 are disposed on the lead. The 1-3-3-1 electrode configuration of FIGS. 5F, 5G, and 5H has two sets of segmented electrodes, each set containing three electrodes disposed around the circumference of the lead, flanked by two ring electrodes (FIGS. 5F and 5H) or a ring electrode and a tip electrode (FIG. 5G). In some embodiments, the lead includes 16 electrodes. Possible configurations for a 16-electrode lead include but are not limited to 4-4-4-4; 8-8; 3-3-3-3-3-1 (and all rearrangements of this configuration); and 2-2-2-2-2-2-2-2.

Any other suitable arrangements of segmented and/or ring electrodes can be used. As an example, arrangements in which segmented electrodes are arranged helically with respect to each other. One embodiment includes a double helix. One or more electrical stimulation leads can be implanted in the body of a patient (for example, in the brain or spinal cord of the patient) and used to stimulate surrounding tissue. The lead(s) may be coupled to an implantable pulse generator (e.g., IPG 14 in FIG. 2) or an external stimulator (e.g., ETS 20 in FIG. 2).

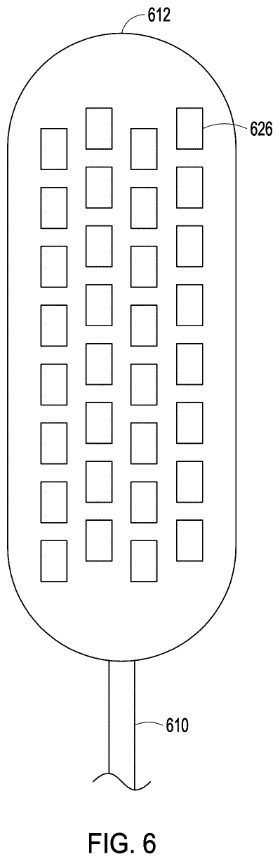

FIG. 6 is an illustration of another example of a lead that is a percutaneous lead sometimes referred to as a paddle lead 612. The paddle lead 612 includes multiple electrodes 626 arranged in multiple parallel rows of electrodes 626. The electrodes 626 are connected to conductor wires in the lead body 610 that are coupled to a neurostimulation device (e.g., IPG 14 or ETS 20 in FIG. 2). In the example of FIG. 6, the electrodes 626 of adjacent rows are offset and staggered in position. Different combinations of electrodes 626 can be selected to steer current to provide different coverage to different stimulation targets. Any of the electrodes 626 can be configured (e.g., through programming) to either be used or unused in the stimulation. When the electrode 626 is used, the electrode 626 can be used as an anode or cathode and carry anodic or cathodic current. Additionally, an electrode 626 may be used as an anode for a period of time and a cathode for a period of time.

When appropriately placed, the stimulation lead 12 delivers stimulation energy (e.g., electrical current) to the tissue of the neurostimulation target and the stimulation produces the intended benefit to the patient with a minimum of side effects. It is also desired to produce the intended benefit with lower stimulation energy. Lower energy can reduce the side effects of the neurostimulation and can extend the battery life of a battery powered neurostimulation device (e.g., IPG of FIG. 1).

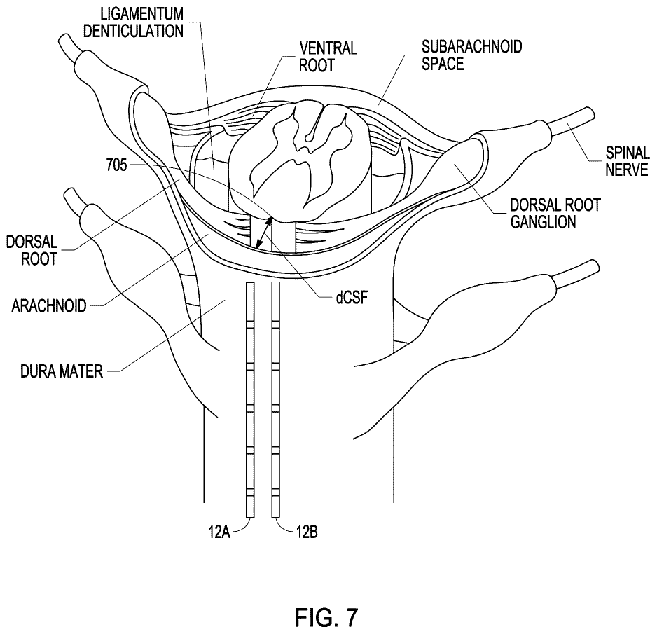

FIG. 7 is an illustration of a portion of a spinal cord and implantable SCS leads 12A, 12B. FIG. 7 also indicates cerebrospinal fluid (CSF). Dorsal cerebrospinal fluid (dCSF) thickness directly affects the energy required to stimulate the neural targets for SCS. Also, scar tissue may form around the SCS lead after it is initially implanted. The scar tissue may also affect the electrical coupling between the SCS device and the intended stimulation target. The dCSF thickness can be measured using Magnetic Resonance Imaging (MRI). However, the dCSF thickness for a patient may vary from the MRI measured data, such as with position or posture of the patient and activity of the patient. The change in dCSF thickness can change the distance or orientation between the stimulation electrodes and the stimulation target, and the powered delivered may not be optimized due to variation in dCSF thickness. Knowledge of the dCSF thickness could be used to adjust the power of the neurostimulation delivered to the patient.

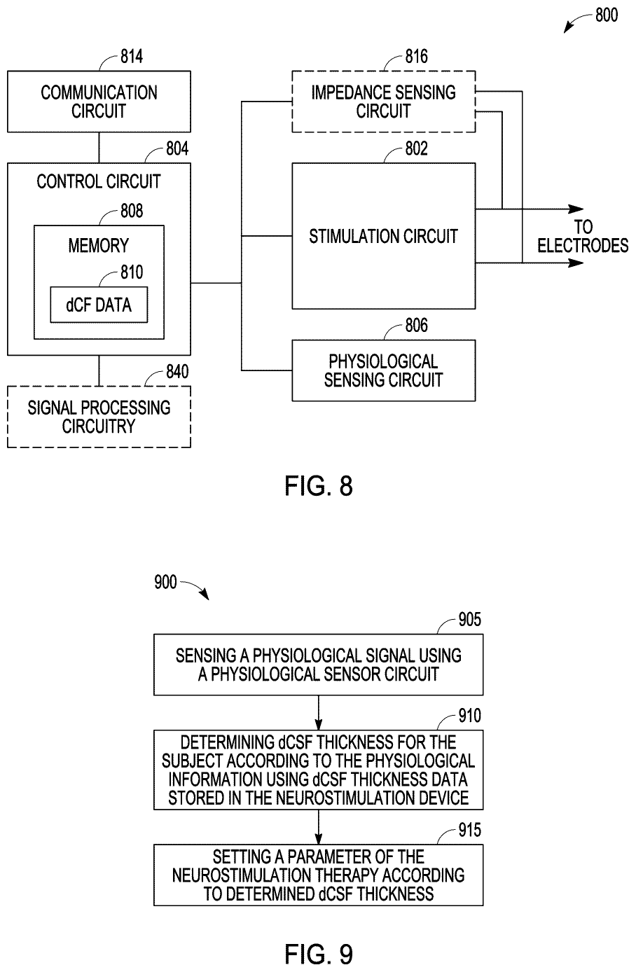

FIG. 8 is a block diagram of portions of an example of a neurostimulation device 800 for providing neurostimulation to a patient or subject. The neurostimulation device 800 may be an IPG (e.g., IPG 14 in FIG. 2) or an ETS (e.g., ETS 20 in FIG. 2). The neurostimulation device 800 includes a stimulation circuit 802, a communication circuit 814, a control circuit 804, and a physiological sensor circuit 806. The stimulation circuit 802 can be operatively coupled to stimulation electrodes such as any of the electrodes described herein (e.g., electrodes 26 in FIG. 2) and the stimulation circuit 802 provides or delivers electrical neurostimulation energy to the electrodes. The communication circuit 814 is configured to communicate with a separate device using a wireless communications link. The control circuit 804 can include a processor such as a microprocessor, a digital signal processor, application specific integrated circuit (ASIC), or other type of processor, interpreting or executing instructions in software modules or firmware modules. The instructions can be stored in memory 808 that can be integral to the control circuit 804 or separate from the control circuit 804. The control circuit 804 can include other circuits or sub-circuits to perform the functions described. These circuits may include software, hardware, firmware, or any combination thereof. Multiple functions can be performed in one or more of the circuits or sub-circuits as desired.

The neurostimulation device 800 includes a physiological sensor circuit 806 configured to produce a sensed physiological signal. The sensed physiological signal provides physiological information regarding the patient. The memory 808 of the neurostimulation device stores dCSF thickness data 810 for the patient or subject. The control circuit 804 deduces the thickness of dCSF of the patient from the physiological information provided by the sensed physiological signal. As discussed previously herein, the dCSF thickness for a patient can vary. The control circuit 804 varies the neurostimulation therapy with variation in dCSF thickness.

FIG. 9 is a flow diagram of an example of a method 900 of operating the neurostimulation device 800 to deliver electrical neurostimulation therapy when connected to an implantable lead (e.g., any of the implantable leads described herein) that includes the electrodes. At block 905, the neurostimulation device 800 senses a physiological signal using the physiological sensor circuit 806. The sensed physiological signal includes physiological information regarding the subject.

In some examples, the physiological sensor circuit 806 is a posture sensing circuit (e.g., a multi-axis accelerometer or a tilt switch). The signal or signals produce by a posture sensing circuit can be used to detect the position of the subject, such as detecting whether the patient is in an upright position, a supine position, a prone position, on their left or right side, or if the patient is in a tilted position.

In another example, the physiological sensor circuit 806 is a signal sensing circuit. The human nervous system produces a neural response to neurostimulation received via sensory receptors or received directly into any part of the network of neural elements that forms the nervous system. Additionally, neurostimulation can excite elements of the human nervous system in a manner that produces neural responses. These neural responses are known as evoked potentials. Evoked potential (EP) signals can be sensed electrically by the neurostimulation device 800, such as by using sense amplifiers of the signal sensing circuit coupled to recording electrodes for example. A repetitive stimulus can be applied or presented to the nervous system and the evoked potential signals (e.g., evoked resonant neural activity (ERNA) signals) sensed from the presentations can be processed (e.g., filtered by averaging) to detect presence of evoked potentials.

The signal sensing circuit is configured to produce sensed Evoked Compound Action Potential (ECAP) signals when coupled to the electrodes. The ECAP signals are EP signals that are the electrical response produced by a group of nerve fibers electrically activated by the neurostimulation device 800. To sense an ECAP signal, the control circuit 804 initiates delivery of electrical neurostimulation energy to target tissue and senses the ECAP signal produced by the neurostimulation.

At block 910, the neurostimulation determines the dCSF thickness for the patient using the physiological information. The memory 808 of the neurostimulation device stores dCSF thickness data 810 for the patient or subject. The dCSF thickness data 810 may be downloaded to the memory 808 from a separate device using the communication circuit 814. In some examples, the neurostimulation device determines the dCSF thickness data 810 as described elsewhere herein and stores the dCSF thickness data in the memory 808. The dCSF thickness data 810 is stored in association with the physiological information, and the control circuit 804 uses the physiological information to deduce the thickness of the dCSF for the patient.

For instance, the dCSF thickness data 810 can be stored in association with the physiological information and a current dCSF thickness can be determined from current physiological information. In an example, if the physiological information is the position of the patient, values of dCSF thickness can be stored in memory 808 for different positions of the patient. From the current position of the patient determined using the posture sensing circuit, the control circuit 804 can deduct the value of dCSF thickness by reading the value of dCSF thickness from memory 808 for that patient position.

In another example, if the physiological information is ECAP signal data, ECAP signals can be evoked and sensed for multiple anatomical locations of the patient. In certain examples, data for multiple ECAP signals can be stored for each anatomical location. Values of dCSF thickness can be measured for the ECAP signals and stored with the ECAP signal data. Using ECAP signal data obtained from a sensed ECAP signal at a particular anatomical location, the control circuit 804 can find the dCSF thickness for the anatomical position by matching the ECAP signal data.

At block 915, the control circuit 804 uses the value of dCSF thickness to set one or more parameters of the neurostimulation therapy. For example, the control circuit 804 may set one or both of the energy level of the neurostimulation therapy and the direction of the neurostimulation therapy energy based on the determined dCSF thickness. The memory 808 may store neurostimulation therapy parameters or sets of parameters for different values of dCSF thickness. The dCSF thickness value deduced by the control circuit 804 can be used as an index to load one or more parameters for the neurostimulation therapy parameters. The control circuit 804 can continuously adjust the neurostimulation therapy based on the physiological information. For instance, as the patient changes position throughout the day, the control circuit 804 deduces the dCSF thickness from the stored data and adjusts the neurostimulation accordingly. The control circuit may also use information regarding scar tissue around the SCS lead to adjust the neurostimulation.

The dCSF thickness data 810 can be downloaded to the memory 808 of the neurostimulation device 800 from a separate device. For instance, the neurostimulation device 800 can be an IPG and the separate device can be a CP. The CP downloads the dCSF thickness data 810 to the IPG (e.g., using communication circuit 814. The IPG can use the dCSF thickness data 810 to adjust neurostimulation therapy it provides to the patient. The dCSF thickness data 810 may be dCSF thickness data obtained for a patient population. In some examples, the dCSF thickness data 810 can be dCSF thickness data for the specific patient receiving the neurostimulation therapy. The dCSF thickness data may be obtained for the patient by the separate device and downloaded to the neurostimulation device.

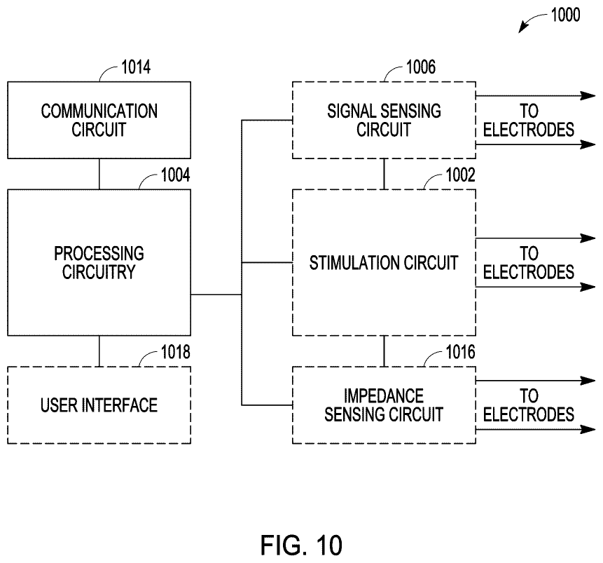

FIG. 10 is a block diagram of portions of a separate device 1000 that determines dCSF thickness data of a patient and provides the data to a neurostimulation device. The device 1000 may be a CP 18, ETS 20, or other external device. The device 1000 includes a communication circuit 1014 and processing circuitry 1004. The communication circuit 1014 is used to communication information with a separate device using a wireless communications link. Processing circuitry 1004 can include processes running on one or more processors (e.g., microprocessors) to perform the functions described. The device 1000 optionally includes a stimulation circuit 1002 to provide electrical neurostimulation energy to the electrodes.

The device 1000 optionally includes an electrical impedance sensing circuit 1016. The impedance sensing circuit 1016 produces a sensed electrical impedance signal when coupled to electrodes. The impedance sensing circuit 1016 may inject current between a pair of electrodes, sense the resultant voltage between the same or different pair of electrodes, and determine impedance, such as by using Ohm's Law, to sense the impedance signals. The processing circuitry 1004 may include signal processing circuitry (e.g., a Digital Signal Processor or DSP) to perform signal analysis or other signal processing on the sensed impedance signals. The processing circuitry 1004 produces electrical impedance image data from the patient. Electrical impedance signals can be sensed at multiple anatomical locations of the patient using the electrodes of one or more implantable leads. Electrical image data can be produced for the anatomical locations using three dimensional (3D) or two dimensional (2D) electrical impedance tomography (EIT). In certain examples, the processing circuitry 1004 receives electrical impedance information from another device. For instance, an IPG 14 may include the impedance sensing circuit 1016 and the device 1000 uploads electrical impedance information measured using the IPG 14.

EIT is a type of medical imaging that uses electrical impedance (or conversely conductance) of bodily tissue to form a tomographic image of the tissue. Changes in bodily tissue within a volume of an EIT stimulation pattern are reflected in impedance changes within the volume. The EIT stimulation pattern can be created using the electrodes of the implantable lead(s). Certain electrodes of the lead are configured (e.g., through programming of the processor circuit 1004) to be active in applying the current to a volume of the spinal cord that includes the CSF, and the other electrodes are configured to sense the voltage equi-potentials resulting from the applied current.

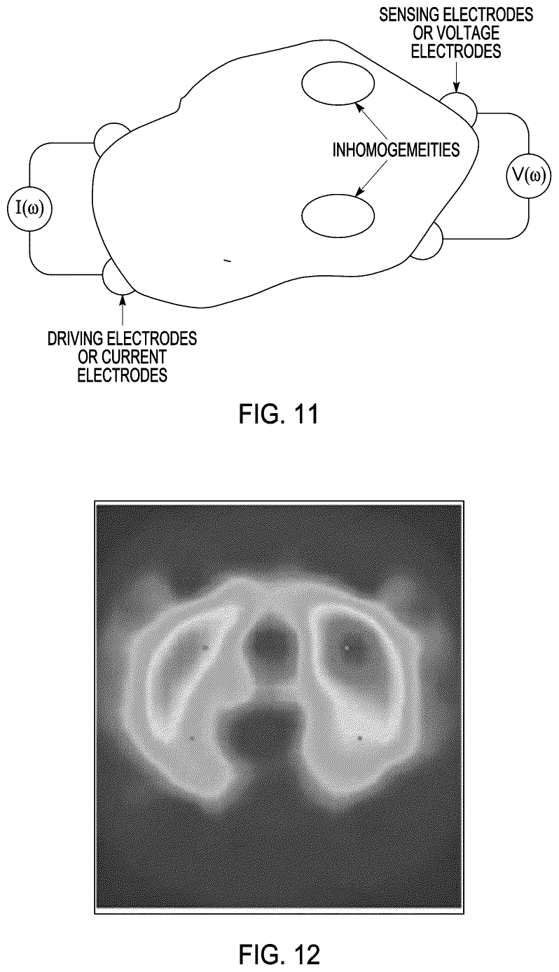

FIG. 11 is a simplified diagram useful to explain the process of producing the EIT image data. The impedance sensing circuit 1016 uses the driving electrodes to inject current I(ω) at the boundary of the epidural space under test according to the EIT stimulation pattern. The impedance sensing circuit 1016 uses the sensing electrodes to sense the voltage V(ω) resulting from the stimulation and the processing circuitry 1004 determines impedance image data from the known current and measured voltage. More than one voltage measurement may be taken, and the impedance determined using one or more of the measured voltages. Inhomogeneities in the tissue will result in inhomogeneities in the measured impedance.

FIG. 12 shows an example of a cross sectional image constructed from the impedance inhomogeneities. The processing circuitry 1004 determines the dCSF thickness data using the impedance image data. The dCSF thickness data can be determined for multiple anatomical locations along the spinal cord of the patient. Also, the dCSF thickness data for an anatomical location can be monitored over time to track changes in the dCSF thickness data for the anatomical location. The dCSF thickness data can be downloaded to the neurostimulation device to use in adjusting neurostimulation therapy. Thus, dCSF measurements can be calculated and stored for a specific patient. The impedance image data may also provide information of scar tissue at the target site. Using one or both of the dCSF information and the scar tissue information can result in more accurate computations of the required electric fields to produce focal stimulation at a certain point in lead-space of the patient, rather than relying on an average of a patient population or relying on an anatomical atlas to decide on a focal stimulation.

In other examples, electrode impedance spectroscopy (EIS) is used to obtain the impedance imaging data. Frequency of the driving current signal is varied, and the sensed impedance is mapped as a function of frequency. Electrical impedance information is collected for each frequency setting and image data may be produced using the electrical impedance information and mapped for multiple frequencies. Inhomogeneities in bodily tissue is detected from inhomogeneities in the impedance versus frequency mapping.

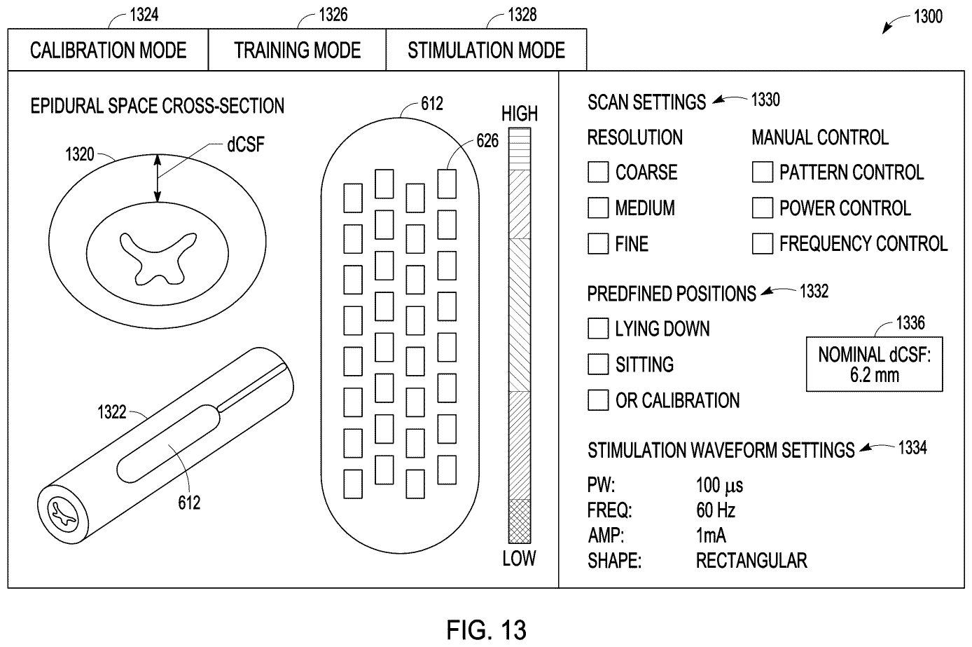

The device 1000 includes a user interface 1018. FIG. 13 is an example of a GUI 1300 for the user interface 1018 to assist the user in determining dCSF thickness data. The GUI 1300 displays a representation of a paddle lead 612 and electrodes 626 used in obtaining the dCSF thickness data. Other types of stimulation leads can be used in the imaging (e.g., four spinal cord stimulation leads 12 of FIG. 2 arranged in parallel). The GUI 1300 also displays a cross section view 1320 of the epidural space (22 in FIG. 11) that is a target for the image, and a 3D view 1322 of the paddle lead 612 and volume of the target epidural space.

The GUI 1300 includes three tabs, including a Calibration Mode tab 1324 for operating the device 1000 in a calibration mode, a Training Mode tab 1326 for operating the device 1000 in a training mode, and a Stimulation Mode tab 1328 for operating the device 1000 in the stimulation mode. The GUI 1300 includes scan settings 1330 for the imaging stimulation pattern. The scan settings 1330 can include resolutions of coarse, medium, and fine, for the resolution of control of the driving and sensing electrodes. The scan settings 1330 can also include manual control for manual setting by the user of the stimulation pattern, stimulation power, and frequency of the stimulation. Features for EIT or EIS paths for dCSF detection can be defined, such as depth of the paths for example, for dCSF detection. The rostrocaudal and resolution value of the depth may also be defined using a coarse, medium, or fine setting.

The GUI 1300 example includes a position menu 1332 for the user to enter the position of the patient for the testing. The dCSF thickness data that is obtained by the device 1000 may then be stored in association with the indicated position. For example, the dCSF thickness information can be stored in the memory of the neurostimulation device together with the position information or the position information can be used as an index for the stored dCSF thickness information. This allows the neurostimulation device to detect the position of the patient (e.g., using a posture sensing circuit) and load the appropriate dCSF thickness information for the detected posture. In the example in FIG. 13, the position menu includes selections for operating room (OR) calibration, the patient sitting, and the patient lying down. Other positions can be included (e.g., standing). The GUI 1300 may also display a nominal dCSF value 1336 for the patient's dCSF thickness.

The GUI 1300 may also include a display of the stimulation waveform settings 1334 for electrical neurostimulation energy provided to the electrodes. The stimulation waveform settings 1334 may be entered using a menu under the Stimulation Mode tab 1328 or another tab.

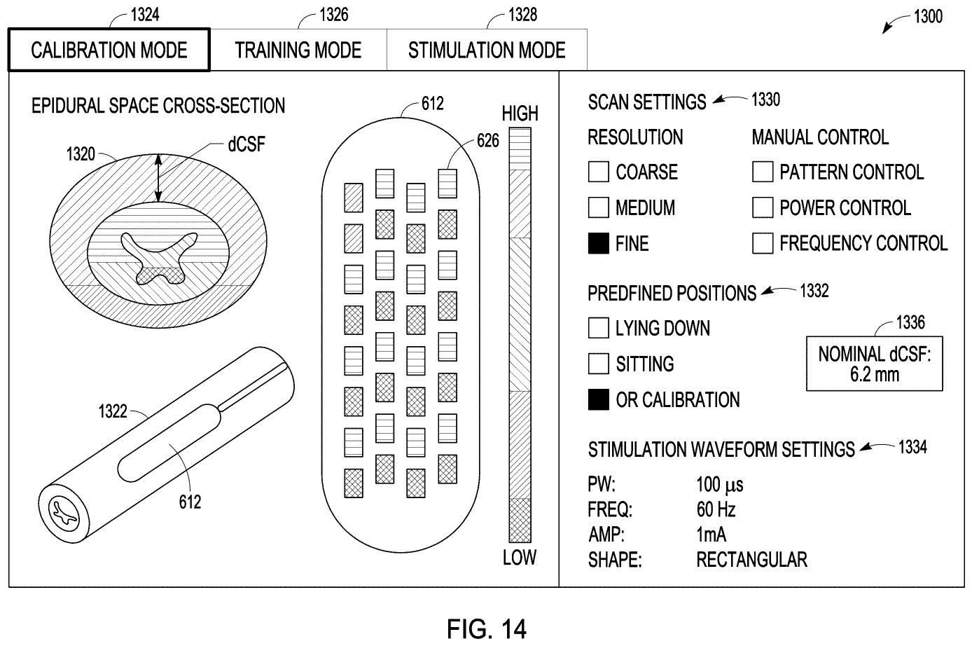

FIG. 14 is an example of the GUI 1300 showing results of a dCSF thickness measurement performed by the device 1000. The scan setting 1330 for the impedance sensing circuit 1016 is set to “Fine.” The display of the paddle lead 612 shows the electrode resolution of the measurements. The highlighted electrodes can be used to indicate the driving electrodes for the measurements. The position menu 1332 indicates OR calibration for the calibration mode. The nominal dCSF value 1336 result is 6.4 millimeters (6.4 mm). Multiple anatomical points of the patient can be imaged by moving the imaging up and down the lead by sequentially changing the rows of electrodes used for driving and sensing up and down the lead. The dCSF thickness can be determined for multiple locations along the lead. This can be done by the processing circuitry 1004 automatically changing the central point of stimulation by changing the combination of electrodes used to obtain dCSF measurements. This automated process may be used to replace the process of tediously obtaining perception threshold measurements to properly initialize therapy or changing amplitude while manually changing or “trolling” the central point of stimulation and manually adjusting amplitude to account for changes in patient perception.

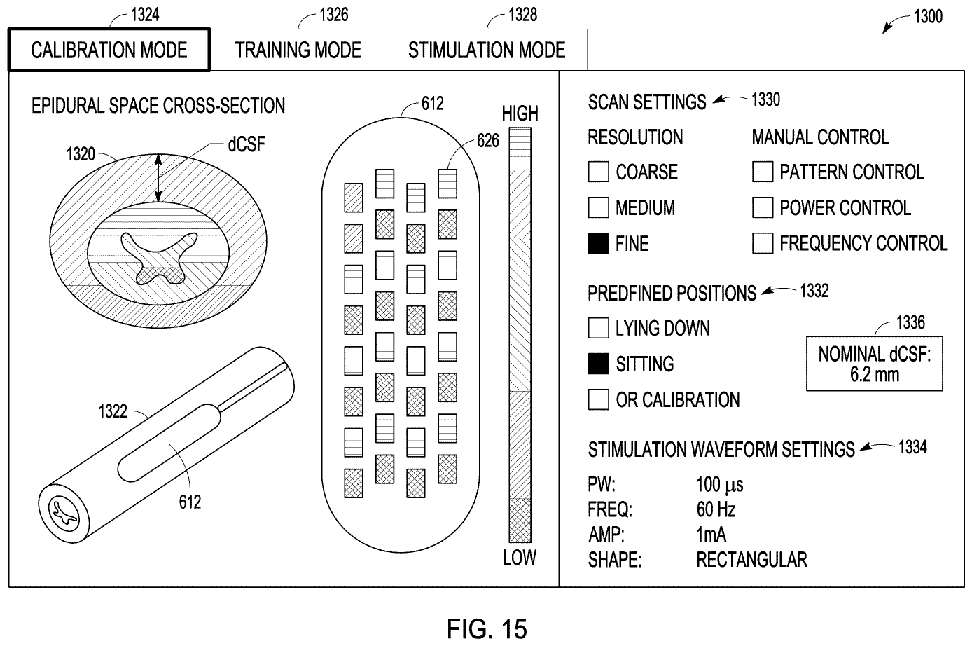

FIG. 15 is an example of the GUI 1300 showing results of a dCSF thickness measurement performed by the device 1000 in the calibration mode for the patient in a sitting position. As shown in the example of FIG. 15, the GUI 1300 may reflect the change in dCSF thickness in a change in the nominal dCSF value 1336 and in a change in position of the structures in the cross section view 1320.

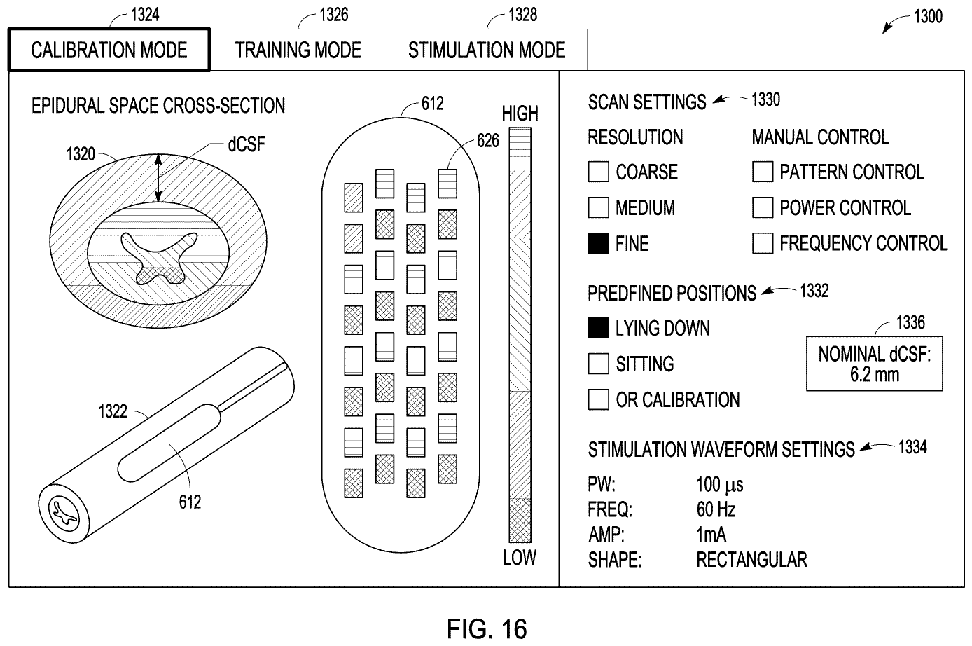

FIG. 16 is an example of the GUI 1300 showing results of a dCSF thickness measurement performed by the device 1000 in the calibration mode for the patient in a lying down position. The change in dCSF thickness is reflected in the nominal dCSF value 1336 and in the change in position of the structures in the cross section view 1320. If there is little variation in the dCSF thickness during calibration, the device 1000 may present a recommendation of not using dCSF thickness to adjust neurostimulation settings. Conversely, if there is sufficient variation in dCSF thickness, the device 1000 may present a recommendation to the user to enable using dCSF thickness to adjust neurostimulation settings. In certain examples, the device 1000 may automatically adjust the neurostimulation settings or program adjusted neurostimulation settings in another device.

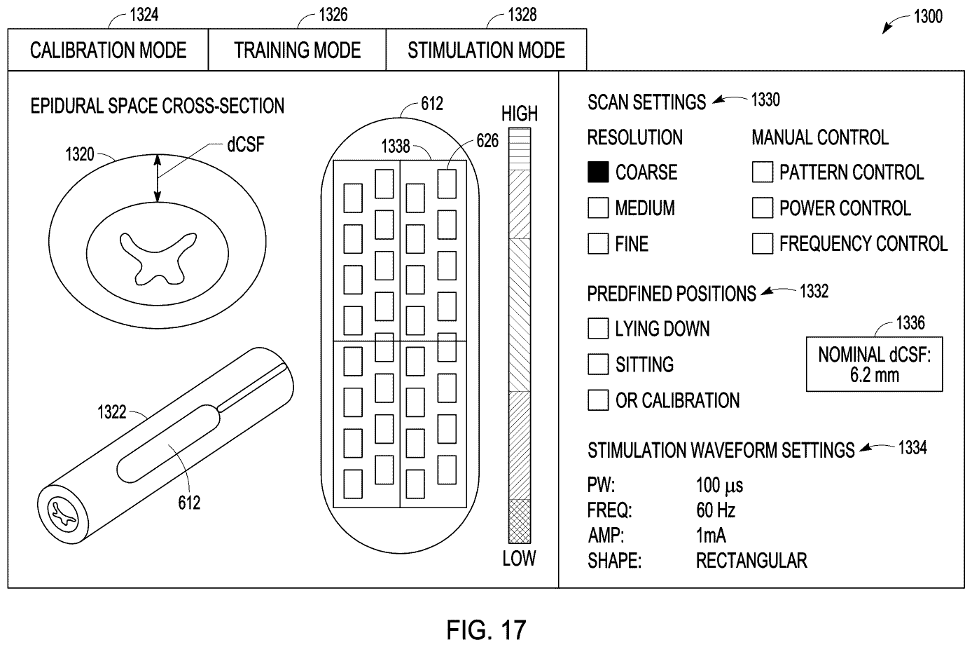

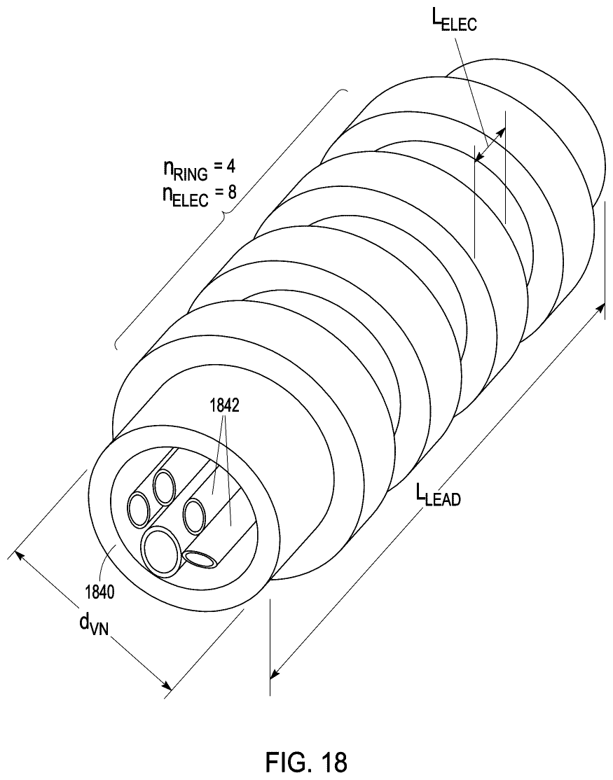

FIG. 17 is an example of the GUI 1300 showing the calibration mode with the scan settings 1330 set to “Coarse.” In the Coarse setting, larger groups of electrodes 1338 are combined for the driving and sensing. The result is lower resolution in the location of the dCSF thickness data. Thickness data for the Coarse setting can be obtained for multiple patient positions.

In the Training Mode, the device 1000 may further process the dCSF thickness data. The dCSF may be measured while the patient is moving (e.g., walking) or changing positions (e.g., changing from left recumbent position to right recumbent position, etc.). The result of the calibration and training modes is a dCSF model for the patient. The training mode may provide improved granularity in the model. In the Stimulation Mode, the device 1000 may run auto-stimulation for the patient (e.g., to manage the patient's pain) with the dCSF data as input for adjusting the neurostimulation. Neurostimulation therapy parameter settings for different dCSF thicknesses may be downloaded to the memory of the neurostimulation to use in adjusting therapy based dCSF thickness deduced by the neurostimulation device.

Returning to FIG. 10, the device 1000 optionally includes a signal sensing circuit 1006 that produces sensed ECAP signals when coupled to the electrodes. The ECAP signals may be evoked in response to electrical neurostimulation energy provided by the stimulation circuit 1002. Values of dCSF thickness can be measured for the sensed ECAP signals and stored in association with ECAP signal data in the neurostimulation device. Using ECAP signal data obtained from a sensed ECAP signal at a particular anatomical location, the control circuit 804 can find the dCSF thickness for the anatomical position by matching the ECAP signal data. This allows the neurostimulation device to load sense ECAP signal data and load appropriate dCSF thickness information for the ECAP signal data.

In some examples, the neurostimulation device itself can obtain the dCSF thickness data. Returning to FIG. 8, the neurostimulation device 800 may be an ETS and optionally includes the impedance sensing circuit 816. The neurostimulation device 800 can include signal processing circuitry 840 to process the impedance signal sensed by the impedance sensing circuit 816. The signal processing circuitry 840 can include a microprocessor, a digital signal processor, application specific integrated circuit (ASIC), or other type of processor. In certain examples, the signal processing circuitry 840 is integral to the control circuit 804. The impedance sensing circuit 816 and the signal processing circuitry 840 may produce the dCSF thickness data 810 using EIT or EIS. The dCSF thickness data 810 can be stored in association with patient position detected using a posture sensing circuit, or in association with ECAP signal data obtained using a signal sensing circuit of the neurostimulation device 800.

Electrical impedance imaging can also be used to monitor for changes in the neurostimulation system. The neurostimulation device 800 may use EIT or EIS to locate a landmark or reference point of the patient's anatomy. For instance, the signal processing circuitry 840 may locate a vertebral landmark or intervertebral space. The distance from one or more leads to the reference point or landmark can be measured using the electrical impedance image data. The signal processing circuitry 840 can recurrently remeasure the distance according to a schedule. This can be useful for the device to detect lead migration over time. Another useful physiological landmark which could be identified with EIT is the dorsal column midline. Knowledge of the midline can assist in programming therapies targeting different anatomical locations. The dorsal column midline can be determined by the region with the greatest dCSF value that continues along the extent of the mapped dorsal column space. The dorsal column midline may also be detected from a drastic change in measured impedance that may reflect a boundary between bone and intervertebral disks. In another example, white matter may be differentiated from gray matter in EIT or EIS images. The dorsal column midline may be inferred from the white matter “crest” 705 indicated in FIG. 7.

The techniques described herein provide in situ imaging for anatomical features of a patient such as the dCSF of the patient using EIT or EIS instead of (or in conjunction with) a more expensive and less convenient procedure such as MRI. The knowledge of dCSF of the patient can be utilized by a neurostimulation device as feedback in adjusting the neurostimulation therapy provided by the device. This customizes the therapy to changes in the dCSF that occur with the movement of the patient.

Imaging other anatomical features using EIT or EIS is also useful. For instance, EIT or EIS can also be used to image peripheral nerves (e.g., the vagus nerve). For neurostimulation, peripheral nerves can be stimulated using a cuff that attaches to the periphery of the nerve and includes multiple electrodes to stimulate the nerve. Imaging peripheral nerves can be used to identify fascicles within a peripheral nerve for targeting peripheral nerve stimulation (PNS). Imaging nerve fascicles can provide information to help select the electrodes to use for stimulation and select parameters for the neurostimulation.

FIG. 18 is an illustration of a stimulation cuff that includes multiple electrodes for stimulation and sensing. The cuff is shown arranged around a vagus nerve 1840 and the cuff may be attached to a stimulation lead. In the example of FIG. 18, the cuff includes 4 rings of electrodes and each ring includes eight electrodes that are configurable as either stimulation electrodes or sensing electrodes. Fascicles 1842 are illustrated within the vagus nerve 1840. The cuff can be used to obtain imaging data using EIT or EIS to map the vagus nerve 1840. The image information can be used to identify the likeliest centroids of the fascicles and relation between the fascicles and the stimulation electrodes.

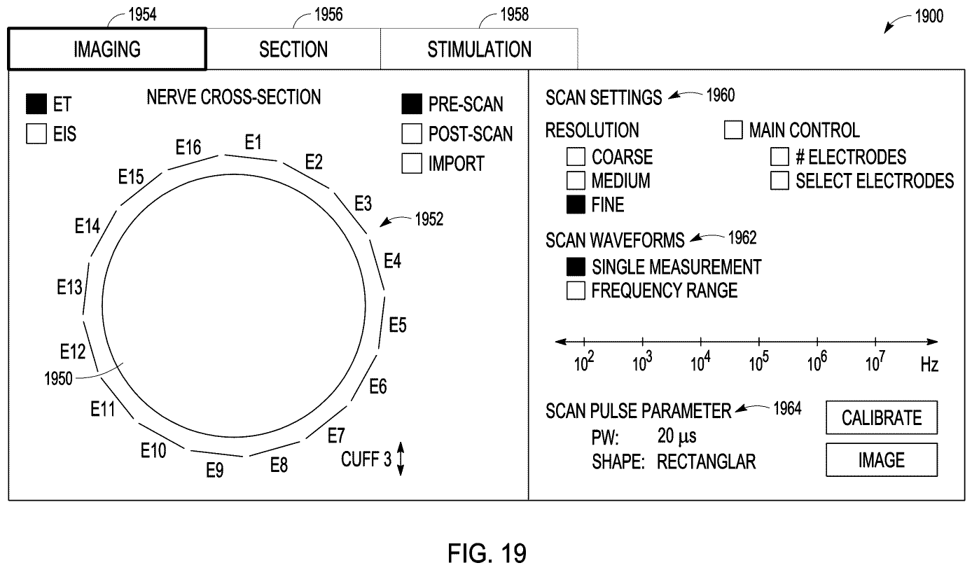

FIG. 19 is an example of a GUI 1900 for the user interface 1018 to assist the user in producing EIT or EIS image data of a peripheral nerve, and to configure neurostimulation for the peripheral nerve using the image data. The GUI 1900 displays a representation of a cross section view of a target peripheral nerve 1950 and a ring 1952 of electrodes of a stimulation cuff. The ring 1952 includes sixteen electrodes labeled E1 through E16. The stimulation cuff may include more than one ring 1952.

The GUI 1900 includes three tabs for subtasks performed by the stimulation device 1000. The tabs include an Imaging Mode tab 1954 for operating the device 1000 to collect EIT or EIS image data, a Selection Mode tab 1956 for selecting stimulation electrodes to stimulate a nerve tissue target, and a Stimulation Mode tab 1958 for operating the device 1000 in the stimulation mode. In FIG. 19, the Imaging Mode tab 1954 is selected. In the Imaging Mode, subthreshold electrical stimulation is applied using selected electrodes to obtain impedance imaging data of the peripheral nerve within the cuff. The subthreshold energy has a high enough amplitude to produce impedance data but not stimulate the target nerve tissue. The GUI Imaging Mode tab 1954 includes scan settings 1960 for the imaging stimulation pattern. The scan settings 1960 can include resolutions of Coarse, Medium, and Fine for the resolution of control of the driving and sensing electrodes. The scan settings 1960 can also include Manual Control for manual setting by the user of the electrodes selected for stimulation.

The Imaging Mode tab 1954 also includes a scan waveform menu 1962. The user can select a single pass of impedance measurements (e.g., for EIT imaging) or the user can select to have multiple passes of impedance measurements made at different frequencies of imaging stimulation (e.g., for EIS imaging). In the example of FIG. 19, a single measurement is selected, and the GUI indicates that tomography imaging (ET 1965) is being performed by the device 1000. The Imaging Mode tab 1954 includes a scan pulse parameter menu in which the user can select the imaging stimulation waveform parameters such as waveform pulse width and waveform shape. The GUI 1900 in FIG. 19 is in a pre-scan mode, and the peripheral nerve has not been scanned for imaging data.

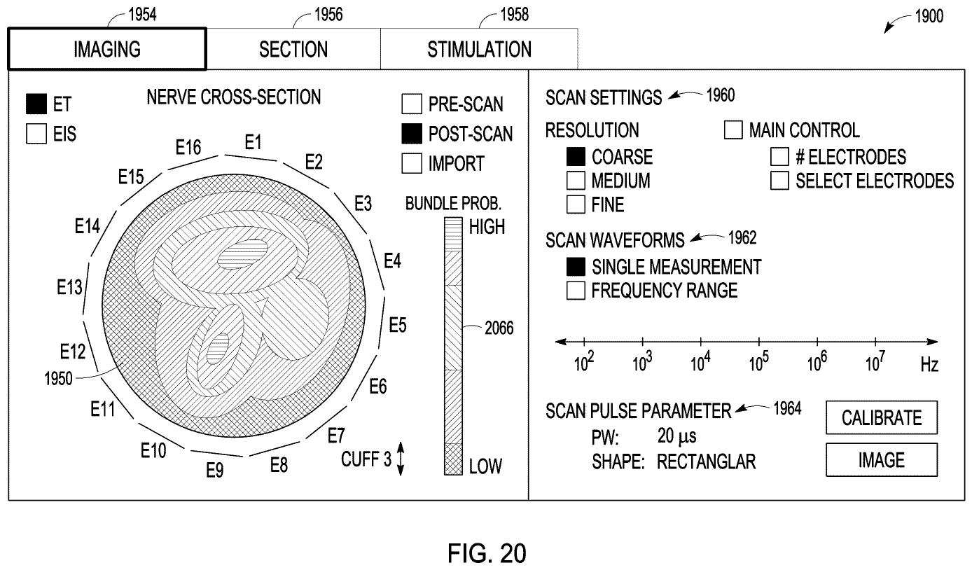

FIG. 20 is an example of the GUI 1900 post scan after impedance imaging data has been produced. The cross section view of the target peripheral nerve 1950 now includes centroids that may represent derived location estimates of the fascicles of the peripheral nerve. Post scan, the GUI 1900 may display a probability scale 2066 to indicate the probability that the centroid shapes are included in a fascicle. The scan imaging was set to the Coarse setting and one pass of scanning was used to collect the impedance imaging data.

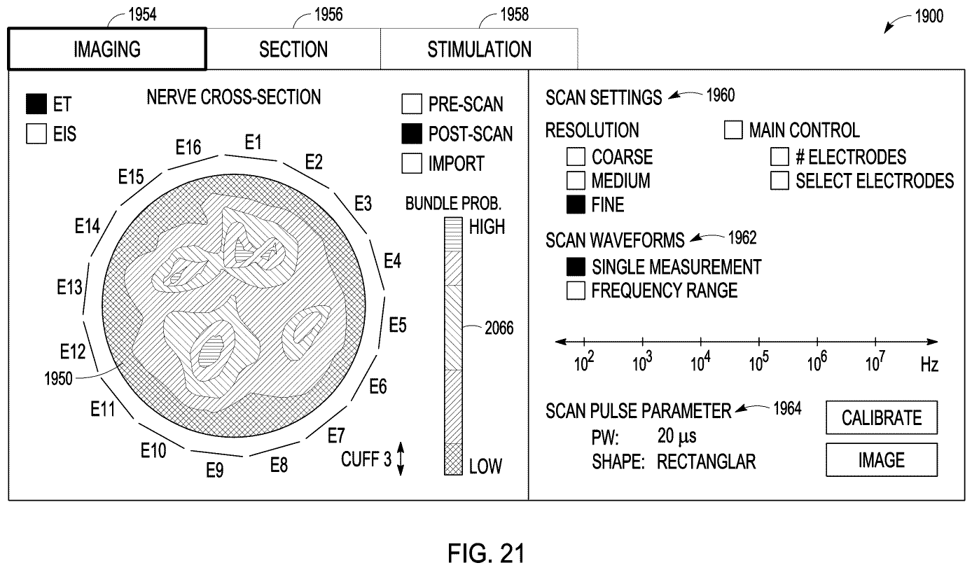

FIG. 21 is another example of the Imaging Mode tab 1954 of the GUI 1900 post-scan. In FIG. 21, the scan imaging was set to the Fine setting, the scanned image includes more detail. The higher probability centroid structures are smaller in the imaging results of FIG. 21.

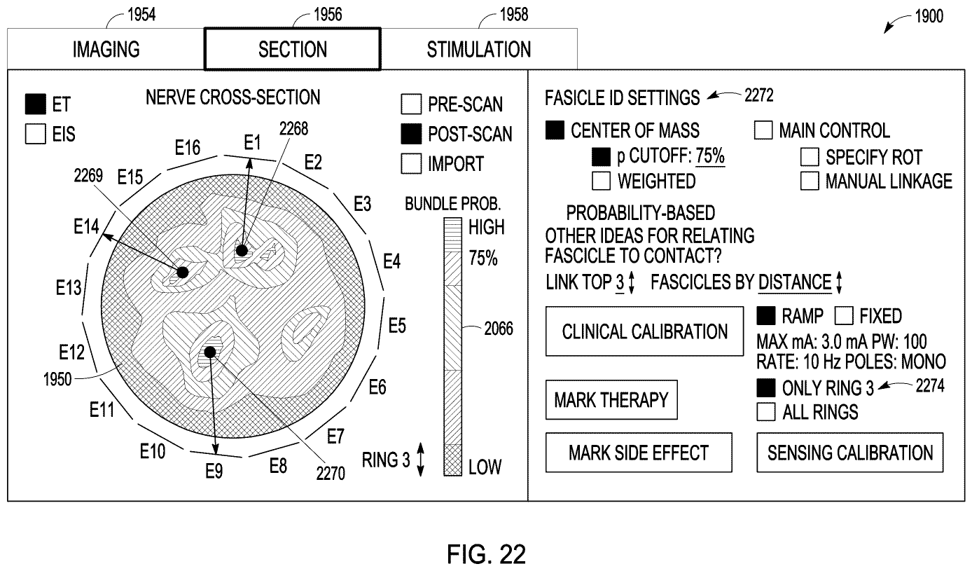

FIG. 22 is an example of a display of the Selection Mode Tab 1956 of the GUI 1900 post-scan after the Fine setting scanning if FIG. 21 is performed. The user may use the Selection Mode Tab 1956 to select electrodes that best target the centroids of fascicles identified in the image view of the target peripheral nerve. For instance, the cross section view of the target peripheral nerve 1950 includes centroids 2268, 2269, and 2270. The user may select electrode E1 as a stimulation electrode for centroid 2268, electrode E14 as a stimulation electrode for centroid 2269 based on proximity of the electrode to the centroid, and electrode E9 as a stimulation electrode for centroid 2270. The Selection Mode Tab 1956 may also include Fascicle ID settings 2272 to adjust identification of the centroids representing fascicles. For example, the Fascicle ID settings 2272 may be used to adjust the rules for identifying centroids to change one or both of the sensitivity of centroids detection and the specificity of centroid detection. The Selection Tab may also include a view menu 2274 to change the display of the target peripheral nerve from a 2D cross section view of one electrode ring to a 3D view of multiple electrode rings. In the example of FIG. 22, the third ring of electrodes is selected for display.

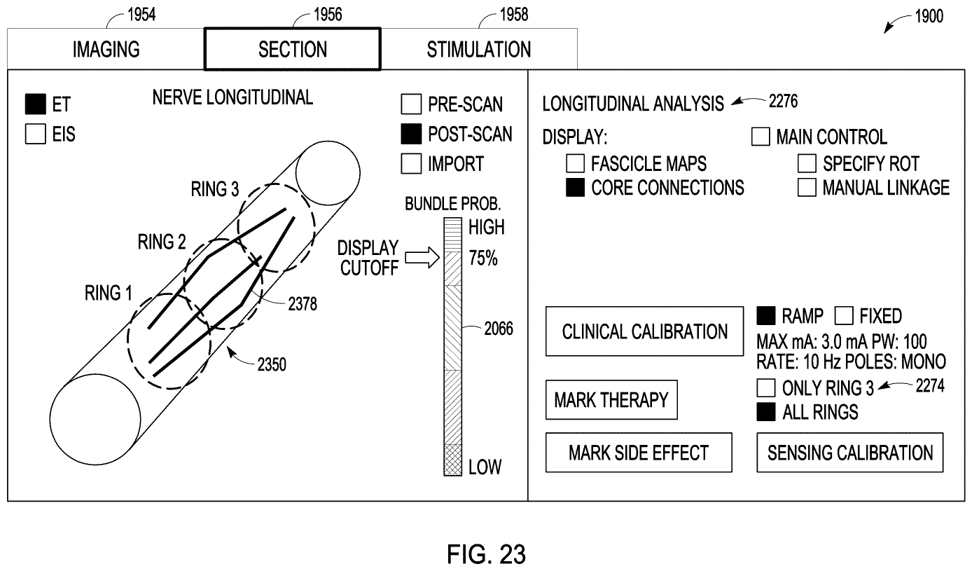

FIG. 23 is an example of a display of the Selection Mode Tab 1956 of the GUI 1900 with all electrode rings of the stimulation cuff selected in the view menu 2274. The stimulation cuff includes 3 rings of electrodes and the view of the target peripheral nerve 2350 is a longitudinal view of the fascicles instead of a cross section view. The Selection Mode Tab 1956 includes a Longitudinal Analysis menu 2376. The Longitudinal Analysis menu 2376 may include a selection to analyze or target a fascicle according to fiber diameter. For instance, the Longitudinal Analysis result may recommend or otherwise identify stimulation electrodes of the electrode rings that target stimulation of an identified fascicle 2378 with the largest diameter.

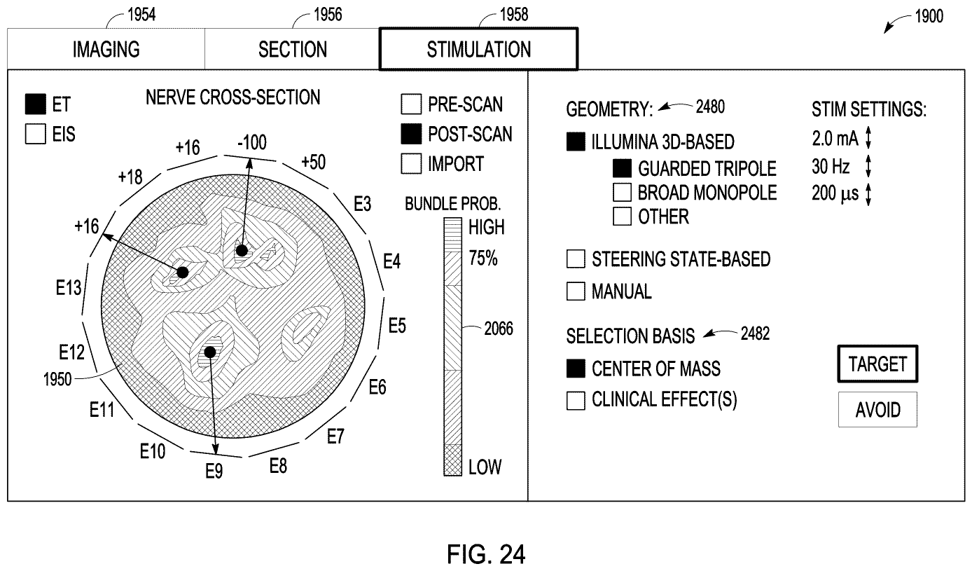

FIG. 24 is an example of a display of the Stimulation Mode Tab 1958 of the GUI 1900. The Selection Mode Tab 1958 allows the user to select electrodes for neurostimulation based on the fascicle location estimates derived from the Imaging and Selection subtasks performed. The display in the example of FIG. 24 is a cross section view of the target peripheral nerve 1950 and the ring electrodes. In the example, ring electrodes E1, E2, E14, E15, and E16 are selected for stimulation. The example shows the fractionalization of the stimulation among the electrodes. The selection and fractionalization may be manually entered by the user using the GUI, or the user can have the device 1000 recommend stimulation electrodes and fractionalization based on parameters entered using the Selection Mode tab 1958. The Selection Mode Tab 1958 can include a stimulation Geometry menu 2480 and a Selection Basis menu 2482. The Geometry menu 2480 can be used to specify the geometry of the stimulation (e.g., the guarded tripole stimulation selected in the example) and the stimulation parameters, such as amplitude frequency and pulse width. The Selection Basis menu 2482 allows the user to select the result based on the best stimulation of the target tissue (e.g., by center of mass) or by avoidance of clinical side effects of the stimulation.

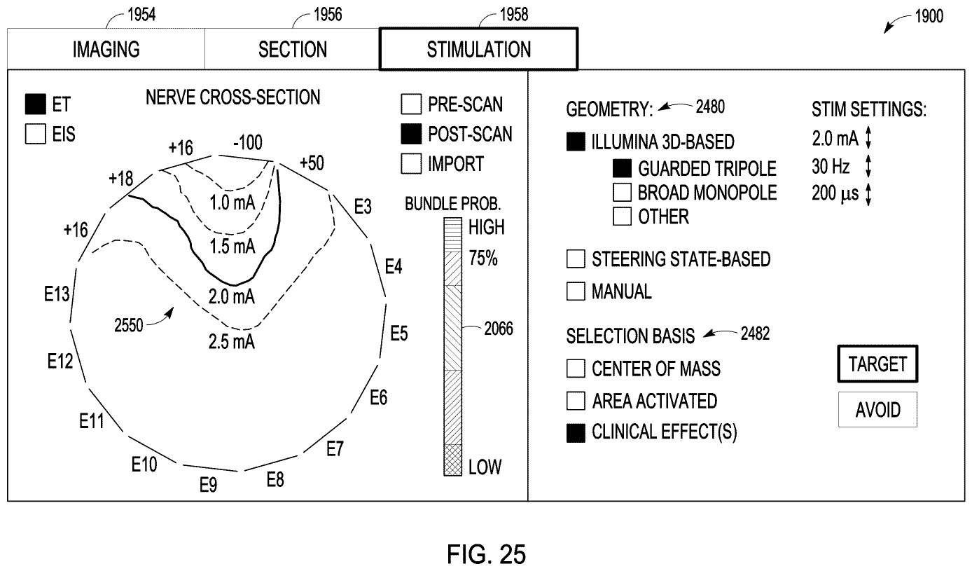

FIG. 25 is another example of a display of the Stimulation Mode Tab 1958 of the GUI 1900. In the example of FIG. 25, the display is a cross section view of the ring electrodes and the fractionalization of the stimulation on the electrodes. But instead of showing the derived estimated fascicle locations of the target peripheral nerve 1950, the extent of the stimulation 2550 relative to the stimulation electrodes is shown for different amplitudes of stimulation current (from 1.0 milliamps (1.0 mA) to 2.5 mA). In certain examples, the display overlays the extent of the stimulation onto the estimated fascicle locations.

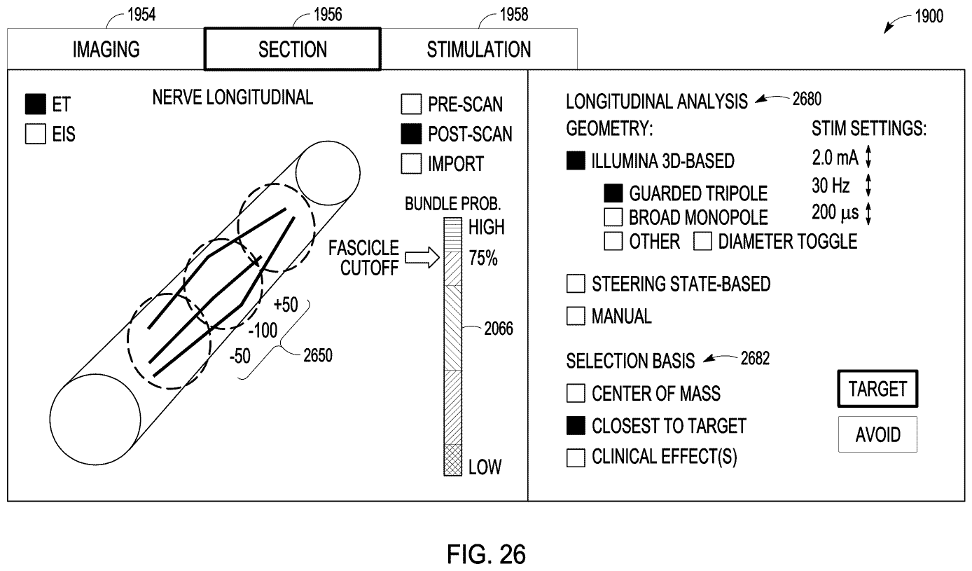

FIG. 26 is an example of a display of the Stimulation Mode Tab 1958 of the GUI 1900 using the longitudinal view instead of the cross sectional view. The example in FIG. 26 shows fractionalization of stimulation by electrode ring instead of by electrode. The fractionalization 2650 may be adjusted according to longitudinal fascicle location estimates derived from the Imaging and Selection subtasks performed by the device 1000. If the electrode rings of the cuff are close enough together, the fiber diameter of the fascicle may be accounted for by adjusting fractionalization on the neighboring rings (e.g., by tightening or widening a tripole stimulation arrangement according to an activation function). The device 1000 may change the Geometry menu 2680 and the Selection Basis menu 2682 when the longitudinal view and analysis is selected by the user. The stimulation geometry and fractionalization may be manually input by the user or the device 1000 derive and present a stimulation configuration to the user. The user may select or adjust the recommended settings.

Returning to FIG. 18, an example of a four-ring stimulation cuff is shown attached to a vagus nerve 1840. An appropriate layout of the electrodes of the stimulation cuff in a grid can lead to the best results of impedance imaging for EIT or EIS. In FIG. 18, some geometric parameters are identified. The diameter of the vagus nerve (d_{VN}) can be the diameter of the stimulation cuff. The active length of the stimulation cuff is L_{LEAD}, the number of rings of electrodes (n_{RING}) is four, and the number of electrodes per ring (n_{ELEC}) is eight. The parameter L_{ELEC} is related to the dimensions of the electrodes.

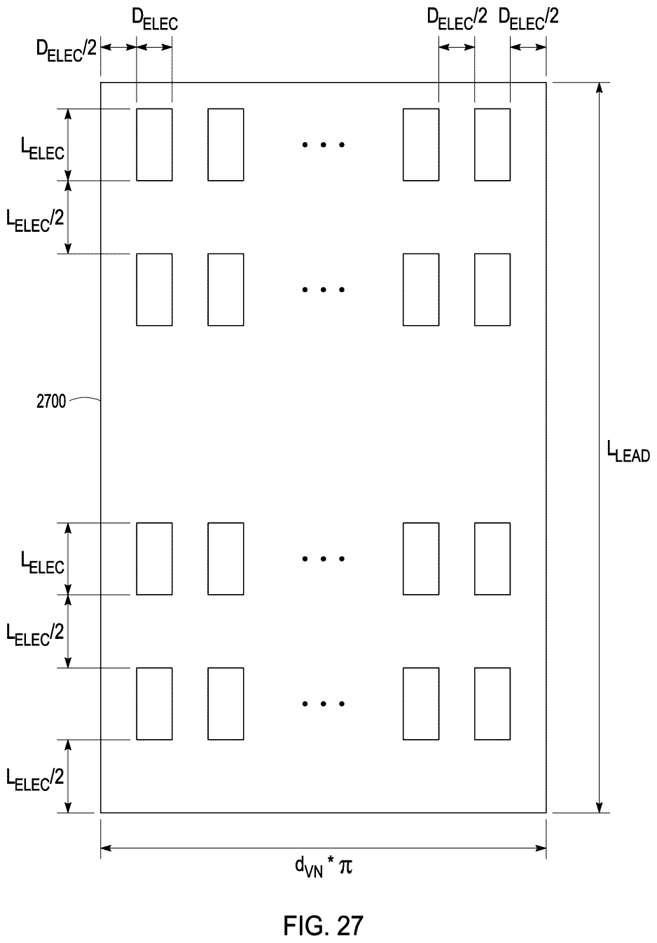

FIG. 27 is an illustration of an example of a grid 2700 for electrode layout for impedance imaging for EIT or EIS. The grid 2700 shows that the parameter L_{ELEC} is the length or height of the electrodes and the length of the spacing between electrodes. The parameter D_{ELEC} is the width of the electrodes. The spacing between electrodes should be at least half of D_{ELEC} (D_{ELEC}/2). The electrodes are arranged in rows and columns. The electrodes include an electrically conductive material and are separated by an electrically insulating material. The length of the grid is thew active length L_{LEAD}, and the width of the grid is d_{VN}*T. The grid 2700 of electrodes is wrapped around the target peripheral nerve along the L_{LEAD} dimension. In certain examples, the electrodes may be included in one sheet as a cylinder in the stimulation cuff, and in certain examples, the electrodes may be included in multiple rings of electrodes as in the example of FIG. 18. The length or height of the electrodes L_{ELEC} can be calculated as

$${L}_{ELEC} = \frac{2 * {L}_{LEAD}}{( 3 * {n}_{RING} + 1 )} ,$$

where n_{RING} is the number of rings of electrodes of the stimulation cuff. The width of the electrodes is related to the width of the grid as

$${D}_{ELEC} = \frac{2 π * {d}_{VN}}{( 3 * {n}_{ELEC} + 1 )} ,$$

where n_{ELEC} is the number of electrodes in a ring of the stimulation cuff. The total number of electrodes T_{ELEC} in the grid 2700 is

$${n}_{RING} * {n}_{ELEC} = {T}_{ELEC} .$$

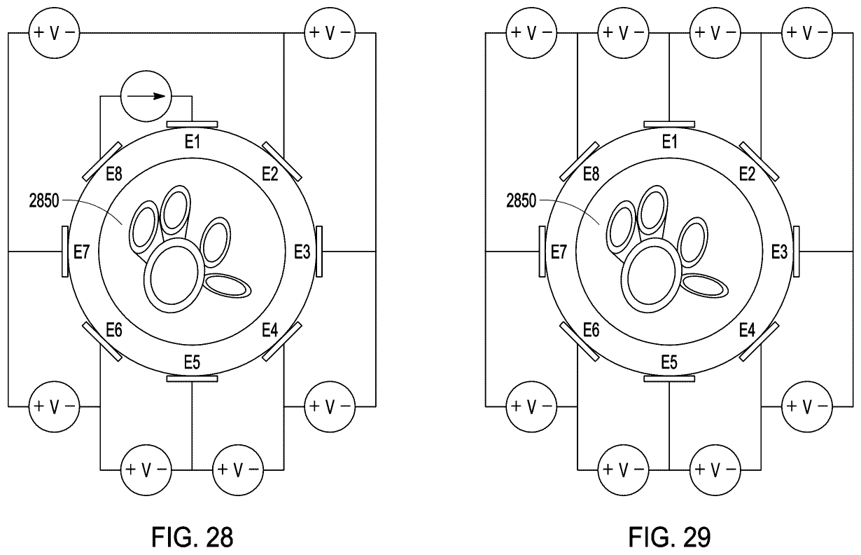

FIG. 28 is an illustration of a cross section of the peripheral nerve 2850 and a ring of the electrodes of the stimulation cuff. The ring of electrodes in FIG. 28 may be any of the four rings of electrodes in FIG. 18. The ring includes eight electrodes labeled E1-E8. Electrodes E1 and E8 are used to apply current to the peripheral nerve and electrodes E2-E6 sense voltage to determine impedance measurements using Ohm's Law. The voltages are sensed between pairs of the E2-E6 electrodes. FIG. 29 is an illustration of a cross section of the peripheral nerve 2850 and another ring of the electrodes of the stimulation cuff. All eight of electrodes E1-E8 are used to sense voltage resulting from the current applied by the E1 and E8 electrodes of FIG. 28. Six voltages are sensed in FIG. 28 and eight voltages are sensed in FIG. 29. If the stimulation cuff includes four rings of electrodes, the remaining two rings are configured to sense voltage as in FIG. 29.

If A is the number of voltage measurements in the electrode ring of FIG. 28, A=n_{ELEC}−2 (where 2 is the number of active or stimulating electrodes), and B is the number of voltage measurements in each of the other electrode rings (B=n_{ELEC}), then the total number of voltage measurements n in the impedance sensing pattern is

$$n = A + ( ( {n}_{RING} - 1 ) * B .$$

In the example of FIGS. 28 and 29, 30 voltages are sensed at different positions along the peripheral nerve. This arrangement may be used for one pass of impedance gathering. Another pair of electrodes from the same or different electrode ring may be chosen as the driving or active electrodes and the other electrodes are used to sense voltages. This can be repeated over the stimulation cuff as desired.

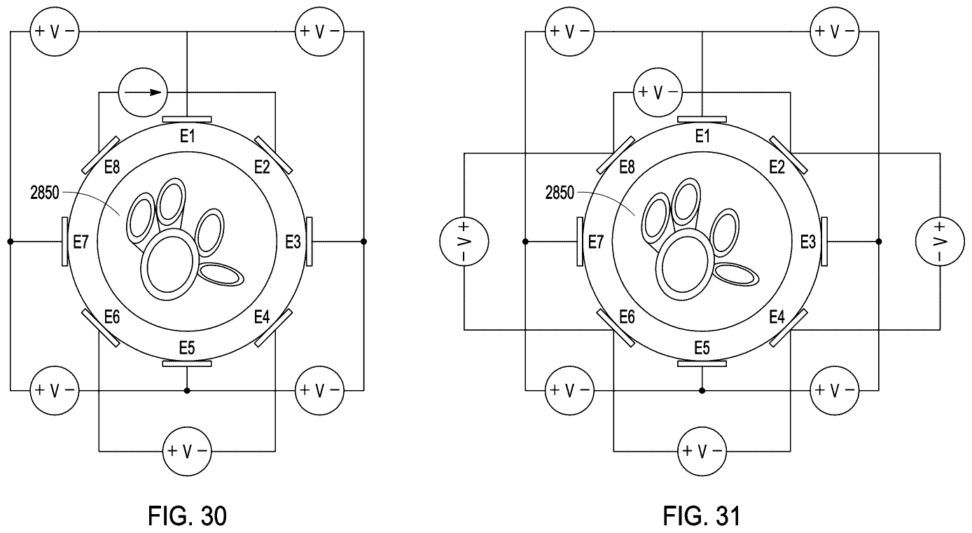

FIGS. 30 and 31 show another pattern of stimulation and sensing to produce impedance imaging data. In FIG. 30, electrodes E2 and E8 are used to apply current to the peripheral nerve and electrodes E1 and E3-E7 are used to sense voltage to determine the impedance. Five voltages are sensed in the example of FIG. 30. FIG. 31 is an illustration of another of sensing using the ring electrodes. Eight voltages are sensed but the electrodes are combined in a different arrangement form the example of FIG. 29. In FIG. 31, adjacent electrodes are not paired for sensing and the electrodes using interleaved pairs. In the example of FIGS. 30 and 31, 29 voltages are sensed at different positions along the peripheral nerve.

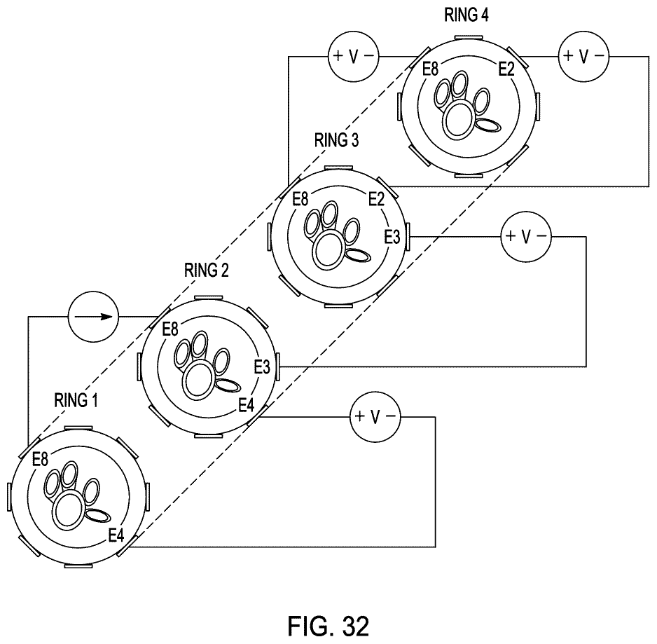

FIG. 32 is an illustration of an example of tangential stimulation and sensing among electrodes of the rings of the stimulation cuff. The fibers of the peripheral nerve run through the four electrode rings. Stimulation current is applied between electrode E8 of RING 1 to electrode E8 of RING 2, so that the stimulation is in a tangential direction along the peripheral nerve. Voltage is sensed between electrode E4 of RING 1 and electrode E$ of RING 2. Voltage is also sensed between electrodes E3 of RING 2 and E3 of RING 3, between electrodes E8 of RING 3 and E8 of RING 4, and between electrodes E2 of RING 3 and E2 of RING 4. Other electrodes of the four rings can be used to sense voltage tangentially between rings or vertically within the same ring.

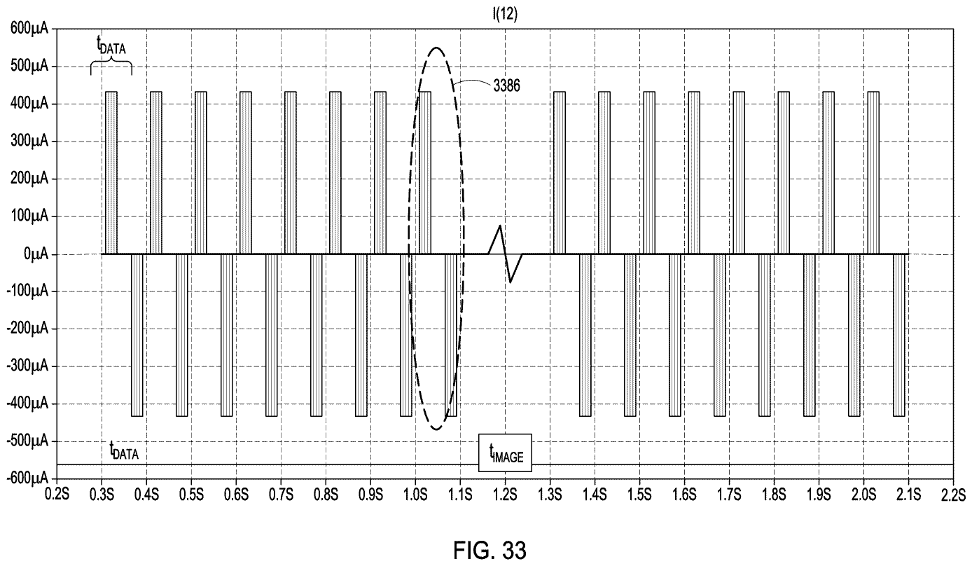

FIG. 33 is a timing diagram of subthreshold stimulation applied to the peripheral nerve to produce impedance image data. The timing diagram shows multiple pulses being applied to the peripheral nerve for imaging. The burst of pulses is delivered during time t_{image}, which is the time used to complete a full image using EIT. Using positive pulse and negative pulses may allow for two images to be obtained at the same time. The collect data time t_{data} is the time needed to collect all differential voltage measurements for one active pair of electrodes.

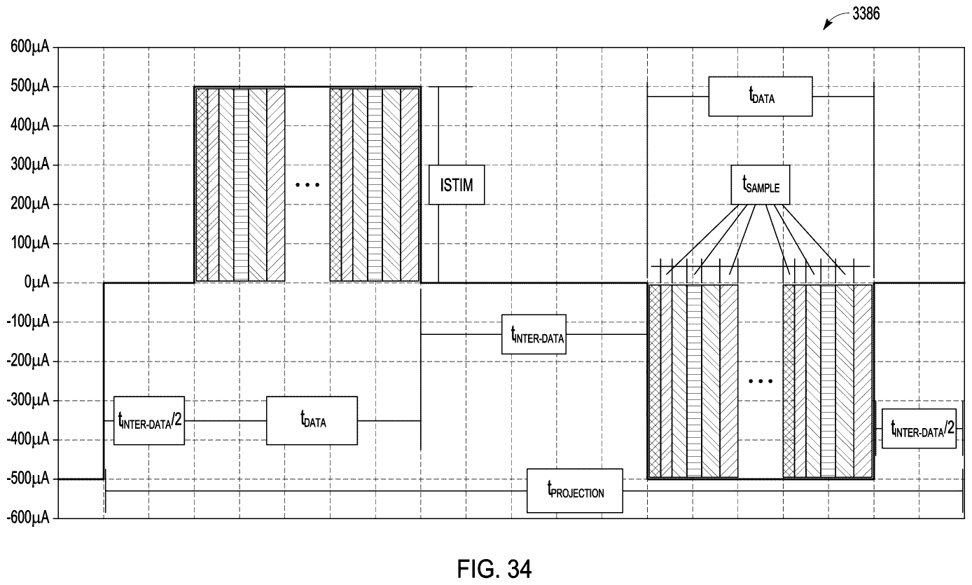

FIG. 34 is an expanded view of the timing of two t_{data} times 3386 of FIG. 33. Time tₛₐₘₚₗₑ is the sample time to make one differential measurement. Each collect data time t_{data} is made up multiple differential measurements. The current Iₛₜᵢₘ is the subthreshold stimulation current applied using the driving electrodes. The stimulating current is applied (e.g., for a pulse width time) and the differential voltages are measured during tₛₐₘₚₗₑ. The time tinter-data is the time between active stimulation pulses being applied to the peripheral nerve. The time t_{projection} is the time needed to finish one image projection. This is also the period of the stimulation or the inverse of the frequency of the stimulation (1/fₛₜᵢₘ).

The techniques described herein for impedance-based imaging enable in vivo mapping of a peripheral nerve. The examples of stimulation cuffs used for imaging may also be used for stimulation by increasing the stimulation above threshold and changing the timing of the stimulation to timing appropriate for neurostimulation therapy. Because the stimulation used for impedance imaging is subthreshold, the mapping produces minimal side effects on the patient. Knowledge of the nerve fascicles gained by the imaging can greatly expedite the time for target searching and can enable informed selectivity.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A neurostimulation device, the device comprising:
a stimulation circuit configured to deliver electrical neurostimulation therapy to a subject when coupled to an implantable lead;
a physiological sensor circuit to produce a sensed physiological signal;
a memory to store dorsal cerebrospinal fluid (dCSF) thickness data for the subject; and
a control circuit operatively coupled to the stimulation circuit, the physiological sensor circuit, and the memory, and configured to:
determine physiological information of the subject using the sensed physiological signal;
determine a value of dCSF thickness according to the physiological information and the stored dCSF thickness data; and
set a parameter of the electrical neurostimulation therapy according to the determined value of dCSF thickness.

2. The device of claim 1,
wherein the physiological sensor circuit is a posture sensing circuit and the sensed physiological signal is a sensed posture signal produced by the posture sensing circuit; and
wherein the control circuit is configured to:
determine position information of the subject using the sensed posture signal; and
determine the value of dCSF thickness using the determined position and the stored dCSF thickness data.

3. The device of claim 1, including:
an impedance sensing circuit configured to produce a sensed electrical impedance signal;
wherein the control circuit is configured to:
produce electrical impedance image data for the subject at multiple anatomical locations using sensed electrical impedance signals;
determine dCSF thickness data for the anatomical locations; and
store the dCSF thickness data for the subject in the neurostimulation device in association with position information of the subject.

4. The device of claim 3, including:
a user interface operatively coupled to the control circuit; and
wherein the control circuit is configured to receive the position information and one or more electrical impedance imaging parameters via the user interface.

5. The device of claim 4,
wherein the control circuit is configured to:
determine a value of variation of dCSF thickness of the subject with posture using the dCSF thickness data; and
present a recommendation of using dCSF thickness to adjust the neurostimulation therapy based on the variation of the dCSF thickness of the subject.

6. The device of claim 1,
wherein the physiological sensor circuit is a signal sensing circuit configured to produce sensed Evoked Compound Action Potential (ECAP) signals when coupled to the implantable lead; and
wherein the control circuit is configured to:
initiate sensing of an ECAP signal at an anatomical location of the subject and determine ECAP signal data using the sensed ECAP signal; and
determine the value of dCSF thickness using the determined ECAP signal data and the stored dCSF thickness data.

7. The device of claim 6, including:
an impedance sensing circuit configured to produce a sensed electrical impedance signal;
wherein the control circuit is configured to:
produce electrical impedance image data for the subject at multiple anatomical locations using sensed electrical impedance signals;
determine dCSF thickness data for the anatomical locations; and
store the dCSF thickness data for the subject in the neurostimulation device in association with the ECAP signal data.

8. The device of claim 1, including:
an impedance sensing circuit configured to produce a sensed electrical impedance signal;
wherein the control circuit includes processing circuitry configured to:
produce electrical impedance image data for the subject at multiple anatomical locations of the subject using electrical impedance tomography (EIT); and
determine the dCSF thickness data for the anatomical locations using the electrical impedance image data.

9. A computer-implemented method of operating a neurostimulation device to deliver electrical neurostimulation therapy when connected to an implantable lead, the method comprising:
sensing a physiological signal using a physiological sensor circuit, wherein the physiological signal includes physiological information of a subject;
determining dorsal cerebrospinal fluid (dCSF) thickness for the subject according to the physiological information using dCSF thickness data stored in the neurostimulation device; and
setting a parameter of the neurostimulation therapy according to determined dCSF thickness.

10. The method of claim 9,
wherein the sensing the physiological information of the subject includes sensing a position of the subject using a posture sensing circuit of the neurostimulation device; and
wherein the determining the dCSF thickness includes determining the dCSF thickness according to the position and the stored dCSF thickness data.

11. The method of claim 10, including:
producing electrical impedance image data for the subject at multiple anatomical locations and multiple positions of the subject using electrical impedance tomography (EIT) or electrode impedance spectroscopy (EIS);
determining dCSF thickness data for the anatomical locations and positions of the subject using the electrical impedance image data; and
storing the dCSF thickness data for the subject in the neurostimulation device in association with patient position.

12. The method of claim 9,
wherein the sensing the physiological information of the subject includes sensing posture and movement of the subject using an accelerometer circuit of the neurostimulation device; and
wherein the determining the dCSF thickness includes determining the dCSF thickness according to the sensed posture and movement and the stored dCSF thickness data.

13. The method of claim 9,
wherein the sensing the physiological information of the subject includes sensing an Evoked Compound Activity Potential (ECAP) signal of the subject using a signal sensing circuit of the neurostimulation device; and
wherein the determining the dCSF thickness includes determining the dCSF thickness according to stored ECAP signal data and the stored dCSF thickness data.

14. The method of claim 13, including:
sensing ECAP signals at multiple anatomical locations of the subject;
determining ECAP signal data for the sensed ECAP signals and storing the ECAP signal data in the neurostimulation device;
determining electrical impedance image data for the subject at the multiple anatomical locations; and
determining dCSF thickness data for the multiple anatomical locations using the electrical impedance image data and storing the dCSF thickness data for the subject in the neurostimulation device in association with the ECAP signal data.

15. The method of claim 9, including:
producing electrical impedance image data for the subject at multiple anatomical locations of the subject;
determining the dCSF thickness data for the anatomical locations using the electrical impedance image data;
determining a variation of dCSF thickness of the subject using the dCSF thickness data; and
producing a recommendation of adjusting the neurostimulation therapy based on the variation of the dCSF thickness of the subject.

16. The method of claim 9, wherein the setting the parameter of the neurostimulation therapy includes setting one or both of power of the neurostimulation therapy and direction of the neurostimulation therapy according to determined dCSF thickness.

17. A neurostimulation programming device, the programming device comprising:
a communication circuit configured to communicate information with a neurostimulation device;
an electrical impedance sensing circuit configured to produce an electrical impedance signal when operatively coupled to electrodes; and
processing circuitry configured to:
produce electrical impedance image data for a patient at multiple anatomical locations of the patient using one of electrical impedance tomography (EIT) or electrode impedance spectroscopy (EIS);
determine dorsal cerebrospinal fluid (dCSF) thickness data for the multiple anatomical locations using the electrical impedance image data; and
communicate the dCSF thickness data to a neurostimulation device.

18. The programming device of claim 17, including:
a user interface; and
wherein the processing circuitry is configured to:
receive position information of the patient via the user interface;
determine the dCSF thickness data for multiple patient positions; and
store the dCSF thickness data for the patient in memory of the neurostimulation device in association with patient position.

19. The programming device of claim 17, including:
a stimulation circuit configured to deliver electrical neurostimulation energy to a subject when coupled to the electrodes;
a signal sensing circuit configured to sense evoked potential signals when coupled to the electrodes;
wherein the processing circuitry is configured to:
initiate delivery of neurostimulation to the subject that produces an Evoked Compound Activity Potential (ECAP);
record sensed ECAP signals at the multiple anatomical locations resulting from the neurostimulation;
determine the dCSF thickness data for the multiple anatomical locations; and
store ECAP signal data in memory of the neurostimulation device in association with the dCSF thickness data.

20. The programming device of claim 17, wherein the processing circuitry is configured to store, in memory of the neurostimulation device, parameters of electrical neurostimulation therapy provided by the neurostimulation device in association with determined values of dCSF thickness.
